(19) United States
(12) Patent Application Publication
(10) Pub. No.: US 2007/0161928 A1
(43) Pub. Date: Jul. 12, 2007

(54) SAMPLING DEVICE FOR IN VIVO SAMPLING OF LIQUIDS FROM THE GASTROINTESTINAL TRACT, PROCESS FOR THE PRODUCTION THEREOF AND MOULD OR MASK FOR USE IN THE PRODUCTION PROCESS

(75) Inventors: Adrianus Joseph Sprenkels, Lelystad (NL); Antonius Johannes Stephanus Maria Jenneboer, Hengelo (NL); Konraad Venema, Driebergen-Rijsenburg (NL); Albert Van Den Berg, Nijverdal (NL); Willem Meindert De Vos, Ede (NL)

Correspondence Address: FOLEY AND LARDNER LLP;SUITE 500, 3000 K STREET NW, WASHINGTON, DC, 20007, US

(73) Assignee: Stichting Technologisch Top-Instituut Voedselwetenschappen

(21) Appl. No.: 11/562,175

(22) Filed: Nov. 21, 2006

(30) Foreign Application Priority Data
Nov. 22, 2005 (EP) 05111070.8

(51) Int. Cl. B65D 81/00; A61B 5/00; G01N 1/16; G01N 1/26

(52) U.S. Cl. 600/575; 600/573; 73/863.31

(57) ABSTRACT

The present invention relates to a sampling device suitable for in vivo sampling of liquid(s) from the gastro-intestinal tract, comprising a body, the body comprising a channel and an opening for entrance of the liquid(s) at one end of the channel, and a cover bonded to at least part of the body and arranged such that the channel in the body is at least partially covered by the cover, the channel having a length of 2 mm-25 m and a perimeter of 2.4-8600 μm, the channel further comprising one or more side compartments, and the channel with the optional one or more side compartments having a volume of 5 nl-4500 μl.

### FIELD OF INVENTION

The present invention relates to a sampling device for in vivo sampling of liquids from the gastrointestinal tract, a process for the production thereof and a mould or a mask for use in the production process.

### BACKGROUND OF INVENTION

Sampling devices for sampling liquids from the GI tract (gastrointestinal tract) are known in the art. WO 02/102243 e.g. describes a sampling device having the shape of a swallowable capsule which allows a sample of a body substance to enter the capsule through an inlet opening which is opened in a predetermined position of the digestive tract following contact with the body substance to be collected. The capsule comprises an elastic blocking member adjacent to the inlet opening wall having a configuration, such that, when the inlet opening has been opened following contact with the body substance, the blocking member has a flow permitting configuration which admits a flow of body substance into a chamber as long as there is a pressure difference between the chamber and the external environment of the capsule and a flow preventing configuration which blocks the inlet opening from the inside of the chamber when the pressure difference has been equalised by the flow of body substance into the capsule.

Another sampling device is described in U.S. Pat. No. 5,971,942. This document discloses a sampler about the size of a medicine pill for oral administration, having a hollow body, an opening, a seal, and a vacuum interior. When the sampler enters the gastrointestinal tract of a patient, the seal is dissolved or disabled and the surrounding fluid is sucked into the sampler by the vacuum inside. The sampler is then recovered from the patient's stool and the gastrointestinal fluid and substances within the sampler can be collected and analyzed.

These prior art documents demonstrate one, relatively large, chamber for sampling and containing the liquid, which means that these devices in reality sample at one place during a short time frame during transport through the GI tract.

Further, analytical devices with channel structures are known from for instance WO 99/46045, WO 2005/020817, US 2004/248306 and US 2002/013457. However, the devices described therein are not suitable for in vivo sampling, or are not swallowable, or are not based on vacuum as driving force or are not suitable or not designed to sample liquids over a longer period of time.

### SUMMARY OF INVENTION

It is an aspect of this invention to provide an alternative sampling device especially for in vivo sampling, with, in yet a further aspect, a design which enables sampling at different places or positions within the GI tract. It is further an aspect of the invention to provide a process for the production of this sampling device. Yet, it is another aspect of the invention to provide a mould or a mask for use in such a process. It is yet another aspect of the invention to provide a method for sampling liquids, in particular from the GI tract. It is again another aspect of the invention to provide a method for analysing liquids, in particular from the GI tract.

According to a first aspect of the invention, there is provided a sampling device especially suitable for in vivo sampling of liquid(s), more in particular for in vivo sampling of liquid(s) from the gastro-intestinal tract, comprising a body, the body comprising a channel and an opening for entrance of the liquid(s) at one end of the channel, and a cover bonded to at least part of the body and arranged such that the channel in the body is at least partially covered by the cover, the channel having a length of 2 mm-25 m and a perimeter of 2.4-8600 μm, the channel optionally further comprising one or more side compartments, and the channel with the optional one or more side compartments having a volume of 5 nl-4500 μl.

According to a second aspect of the invention, there is provided a process for the production of a sampling device for sampling liquid(s), especially from the gastro-intestinal tract, comprising a) providing a channel and an opening for entrance of the liquid(s) at one end of the channel in a body and optionally providing one or more side compartments to the channel, the channel having a length of 2 mm-25 m and a perimeter of 2.4-8600 μm, and the channel with the optional one or more side compartments having a volume of 5 nl-4500 μl, and b) binding a cover to at least part of the body such that the channel in the body is at least partially covered by the cover.

According to another aspect of the invention, there is provided a mould or a mask for use in the process according to the invention.

According to yet another aspect of the invention, there is provided a method for sampling liquid(s) from the GI tract comprising swallowing the sampling device according to the invention.

According to a further aspect of the invention, there is provided a method for analyzing liquid(s) from the GI tract of a human or an animal comprising swallowing the sampling device according to the invention by a human or an animal, retrieval of the sampling device from the GI tract of the human or animal, optionally extracting the liquid from the sampling device, and analyzing at least part of the liquid sampled by the sampling device with analysis techniques known per se.

According to yet another aspect of the invention, there is provided a sampling device, especially for in vivo sampling of liquid(s) from the gastro-intestinal tract, comprising a body, the body comprising a channel and an opening for entrance of the liquid(s) at one end of the channel, and a cover bonded to at least part of the body and arranged such that channel of body is at least partially covered by cover, the channel having a length of 2 mm-25 m and a perimeter of 2.4-8600 μm, the channel optionally further comprising one or more side compartments, and the channel with the optional one or more side compartments having a volume of 5 nl-4500 μl, wherein the device is arranged to sample the liquid(s) into the channel and the optional one or more side compartments substantially by means of a vacuum force.

In a specific embodiment, the channel in the device of the invention further comprises hydrophobic barriers to favour a flow of the liquid to be sampled in the direction of the side compartment.

Hereby, the invention provides a kind of a liquid sample memory chip (chemical memory chip), designed to sample the liquids over a period of time, preferably by means of vacuum force as driving force for sampling.

### BRIEF DESCRIPTION OF FIGURES

Embodiments of the present invention will now be described, by way of example only, with reference to the accompanying schematic drawings in which corresponding reference symbols indicate corresponding parts, and in which:

FIG. 1 schematically depicts a top view of an embodiment of the sampling device of the invention with a channel;

FIG. 2 schematically depicts a top view of an embodiment of the sampling device of the invention with a channel and side compartments;

FIG. 3 schematically depicts a perspective view of an embodiment of the sampling device of the invention with a channel and side compartments in more detail;

FIG. 4 schematically depicts a perspective view of an embodiment of the sampling device of the invention with a barrier in a channel;

FIG. 5 schematically depicts a side view of an embodiment of the sampling device of the invention with a cover;

FIG. 6 schematically depicts a side view of another embodiment of the sampling device comprising a body and a cover, the body and cover being connected to each other;

FIG. 7a schematically depicts a perspective view of an embodiment of a tablet containing the sampling device according to the invention. FIGS. 7b and 7c schematically depict front views of an embodiment of a tablet containing the sampling device according to the invention.

FIGS. 8a and 8b schematically depict top views of embodiments of the sampling device of the invention with a channel and without (a) or with (b) side compartments.

FIGS. 9a-9d schematically depicted specific embodiments of meandering structures and details thereof according to embodiments of the invention.

FIG. 10 displays a SEM picture of a black silicon hydrophobic barrier.

### DETAILED DESCRIPTION OF EMBODIMENTS

The device of the invention may be used in situ and ex situ. In a particular embodiment, the sampling device of the invention is used for sampling liquids from the GI tract. The device is herein further described with specific reference to use as sampling device for GI liquids. However, the device may also be used for other purposes. Hence, when referring to sampling of GI liquids, the invention is to be understood not to be limited to this specific embodiment. Hence, the sampling device according to the invention may be arranged, especially the channel dimensions, optional side compartment dimensions and side compartment channels dimensions, may be selected to sample GI liquids, but may in other embodiments also be arranged (especially the channel and optional side compartment and side compartment channels dimensions) to sample other (bodily) liquids like blood, waste water, oil, etc.

The term “GI liquid(s)” refers to liquids which can be found in the gastrointestinal tract of humans or animals having a GI tract. GI liquids are e.g. found in the mouth, gullet, stomach, small and large intestine. Such liquids may contain e.g. species in solution (or suspension) such as dietary components, drugs, food components, digestion products, microbial metabolites, etc., of which the presence, concentration, may be monitored by sampling GI liquids according to the method and with the device of the invention. The liquid(s) may further contain one or more gases selected from for instance O₂, H₂, CO₂, CH₄, H₂S, etc. The term “liquid” may also refer to a number of liquids, e.g. to liquid sampled by the device of the invention along the GI tract such as saliva, stomach fluid and intestinal fluid(s). The term “GI liquid(s)” especially refers to “GI liquids”, as known to the person skilled in the art. However, the use of the term “GI liquid(s)” is also exemplary, since also other liquids may be sampled.

The term “swallowable” refers to the dimensions of the device, especially of the device in a form to be swallowed (e.g. included in a tablet or on a carrier) which can e.g. be swallowed by a predetermined group of humans (e.g. a number of adults such as 10 adults of 25-75 years) or animals (for example a number of animals such as 5 beagle dogs of 1 year). This means that the term “swallowable” may be related to a predetermined group of patients, such as adults, children, etc. As will be clear to the person skilled in the art, the term “swallowable” refers to dimensions of the device or of an assembly of the device in a container or on a carrier (like a tablet) which dimensions are selected such that the device or assembly, respectively is suitable for oral administration for one or more target persons (adult, child, etc.).

The term “plain” in connection with the channel refers to embodiments of the invention wherein the channel does not comprise side compartments, i.e. one “long” channel. The term “compartment embodiment” refers to embodiments of the invention wherein the channel comprises in addition to a channel, also one or more side compartments, wherein the side compartments are (in an embodiment) designed to sample a GI liquid of at least about 2 nl (see also below). In both embodiments, the channel (i.e. the main canal) may be straight or bent (in an embodiment the channel may be meandering).

The term “depth” or “height” in relation to a channel or e.g. a side compartment are used interchangeably and mean the same, unless stated otherwise.

The term “channel structure” refers to a channel according to the invention with optional side compartments (and optional side channels), i.e. the whole part of the device that is used for sampling GI liquid(s), including an optional reservoir and connecting channel between the optional reservoir and the channel. Hence, the term “channel structure” refers to all cavities introduced to a body, e.g. by a lithographic process, as depicted in the figures.

An “assembly” indicates a device integrated in a container or on or in a carrier (such as for instance a tablet, a capsule, etc.).

The phrase “the channel with the optional one or more side compartments having a volume of 5 nl-4500 μl” indicates the volume of the channel, including all optional side compartments.

The term perimeter of a channel refers to the circumferential length of a cross-section of the channel. Since most channels of the invention will substantially comprise a rectangular cross-section, the perimeter is in such cases the sum of twice the width and twice the height. The perimeter can be determined assuming a cover is present, since the channels are bounded by two side walls, a bottom surface and a top surface, the top surface being provided by the cover.

Device General

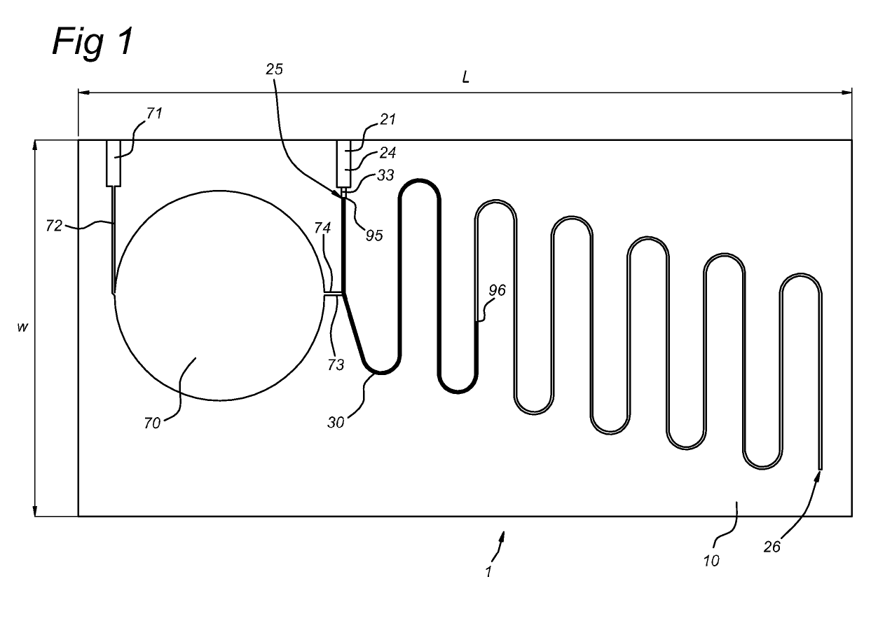

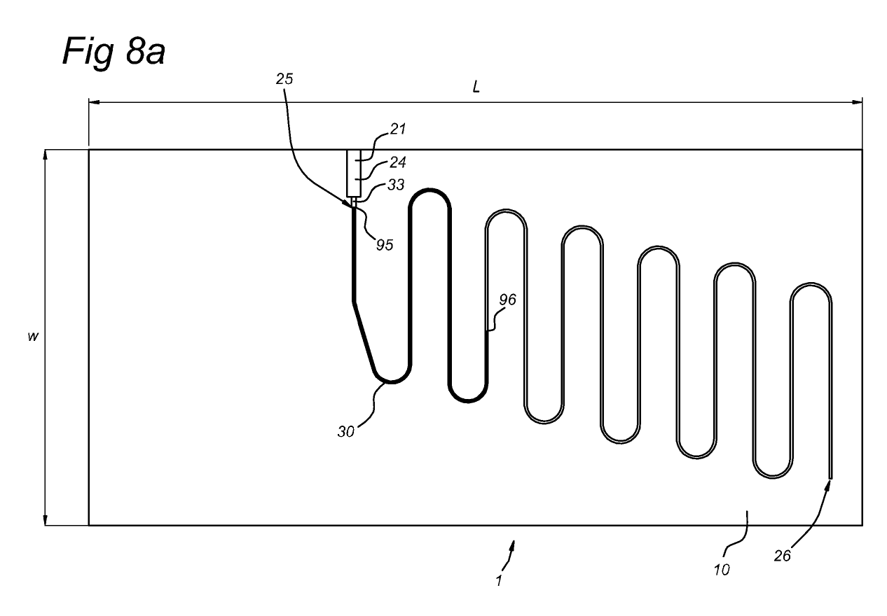

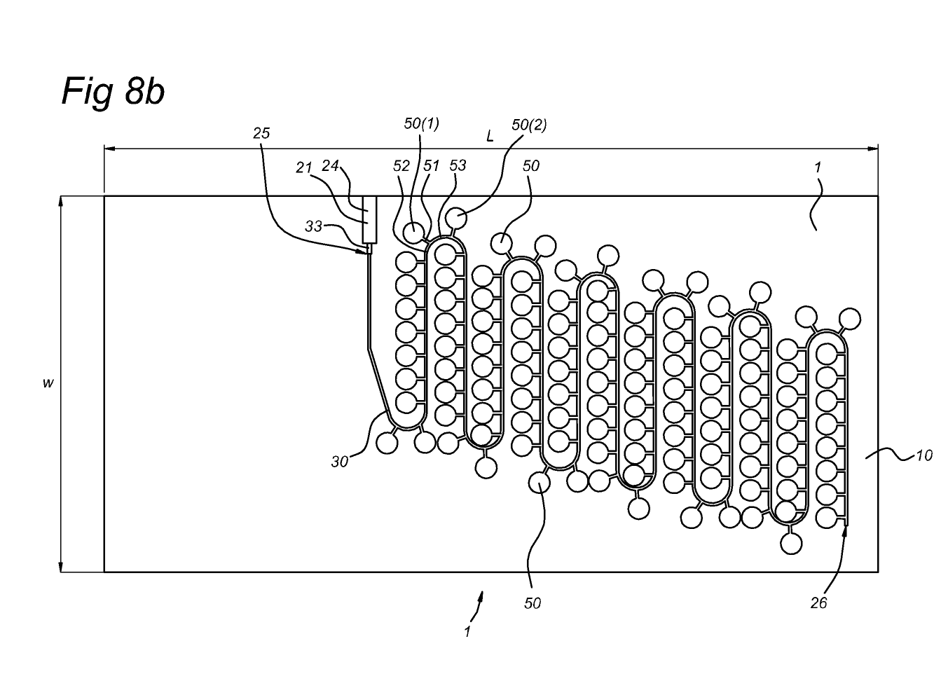

This part of the description describing the device in general applies to both the plain channel and compartment embodiments. In FIGS. 1 and 8a an embodiment of a sampling device 1 for in vivo sampling of liquid(s) from the gastro-intestinal tract according to the invention is schematically depicted.

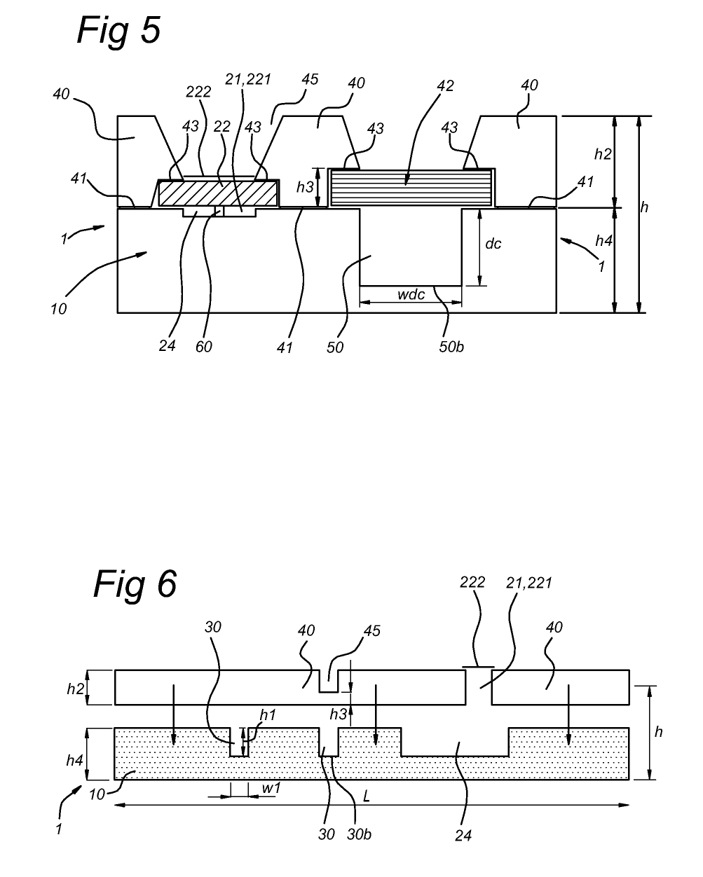

Device 1 has a length L and a width w. The dimensions of device 1 are preferably selected such that device 1 is swallowable, or that an assembly (device 1 contained or arranged in a container or on a carrier, such as e.g. a tablet) has dimensions such that the assembly is swallowable. Hence, length L of device 1 is preferably about 3 cm or smaller, e.g. about 0.5-3 cm, and width w and height h (see FIGS. 5-6) are preferably about 1.5 cm or smaller, e.g. about 0.2-1.5 cm. In a preferred embodiment, the total volume of device 1 is about 0.01-8 cm³, more preferably 0.02-6.75 cm³.

The dimensions may be selected in accordance with the use, e.g. smaller dimensions for sampling of GI liquids of infants and larger dimensions for applications involving adults. Since device 1 may also be used to sample liquids from animals, the above (and below) described dimensions may deviate for e.g. relatively large or small animals, as will be clear to the person skilled in the art.

The device comprises a body 10 wherein a channel 30 and an opening 21 for entrance of the liquid(s) at one end 25 (first end) of channel 30 is provided. Device 1 further comprises a cover 40 (not shown in FIG. 1) bonded to at least part of body 10 and arranged such that channel 30 of body 10 is at least partially covered by cover 40. Examples of covers are shown in FIGS. 5 and 6.

In general, channel 30 may have a length of about 2 mm-25 m and a perimeter of about 2.4-8600 μm. In FIG. 1, the length of channel 30 is the length between a first end 25, i.e. the place where liquid enters channel 30, and second end 26 of channel 30. Referring to FIG. 6, a side view of an embodiment of device 1 is shown with channel 30, having a width w1 and a height h1. Herein, the perimeter of channel 30 is a summation of 2*w1 and 2*h1. In an embodiment, the perimeter and length of the channel and the material of body 10 and cover 40 are selected such that capillary forces can be used for sampling liquids from the GI tract. The range where capillary forces become relevant is dependant upon e.g. liquid properties and the surface properties of the channel walls, as will be known to the person skilled in the art. As a rule of thumb, capillary forces can at least partly be used as driving mechanism with h1 channel height values equal to or below about 2 micron, more preferably equal to or below about 1 micron, even more preferably for channel height h1 equal to or below about 0.5 micron.

In an embodiment, the width w1 of channel 30 is about 1-300 μm, preferably about 10-100 μm. In yet another embodiment, height h1 of channel 30 is about 0.2-4000 μm, preferably about 0.5-300 μm.

In the table below, some embodiments are summarized:

TABLE 1
Example of dimensions for a number of general embodiments^{a}

| | | | | | Minimum | | Maximum | |
|---|---|---|---|---|---|---|---|---|
| | | | | | value of | | value of | |
| | Minimum | | Maximum | | preferred | | preferred | |
| | value of an | | value of an | | embodi- | | embodi- | |
| | embodiment | | embodiment | | ments¹ | | ments² | |
| Length | swallowable | | swallowable | | 0.5 | cm | 3.0 | cm |
| L of | | | | | | | | |
| device 1 | | | | | | | | |
| Width | swallowable | | swallowable | | 0.2 | cm | 1.5 | cm |
| w of | | | | | | | | |
| device 1 | | | | | | | | |
| Height | swallowable | | swallowable | | 0.2 | cm | 1.5 | cm |
| h of | | | | | | | | |
| device 1 | | | | | | | | |
| Length | 2 | mm | 25 | m | 0.01 | m | 10 | m |
| channel | | | | | | | | |
| of general | | | | | | | | |
| embodi- | | | | | | | | |
| ment | | | | | | | | |
| Width w1 | 1 | μm | 300 | μm | 10 | μm | 100 | μm |
| channel | | | | | | | | |
| of | | | | | | | | |
| general | | | | | | | | |
| embodi- | | | | | | | | |
| ment | | | | | | | | |
| Height h1 | 0.2 | μm | 4000 | μm | 0.5 | μm | 300 | μm |
| channel of | | | | | | | | |
| general | | | | | | | | |
| embodi- | | | | | | | | |
| ment | | | | | | | | |
| Volume | 5 | nl | 4500 | μl | 10 | nl | 300 | μl |
| channel | | | | | | | | |
| of general | | | | | | | | |
| embodi- | | | | | | | | |
| ment | | | | | | | | |

^{a}applying to both general embodiments of a plain channel without side compartments and a channel with side compartments
¹other (more) preferred embodiments may have larger minimum values
²other (more) preferred embodiments may have smaller maximum values

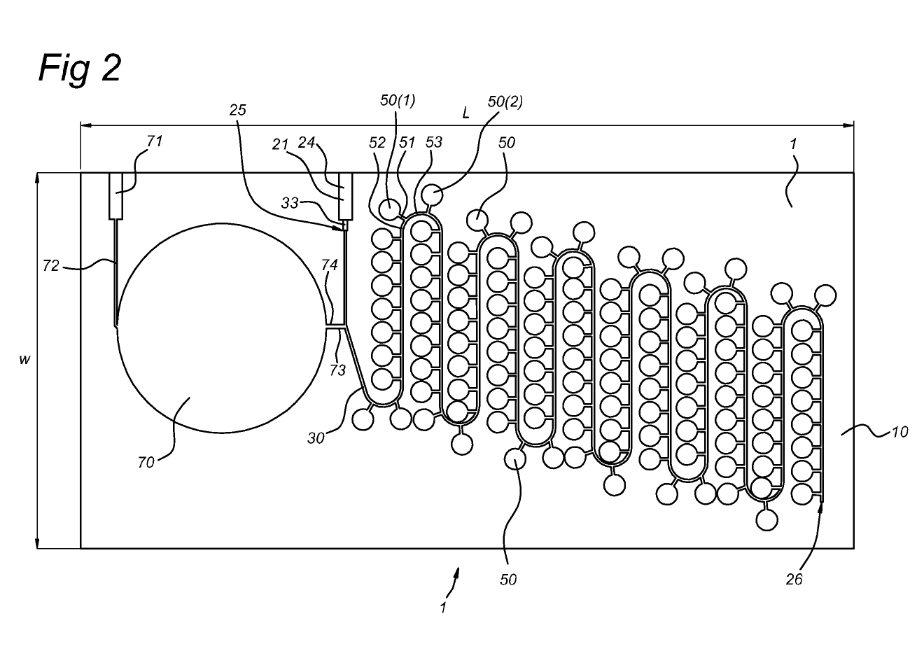

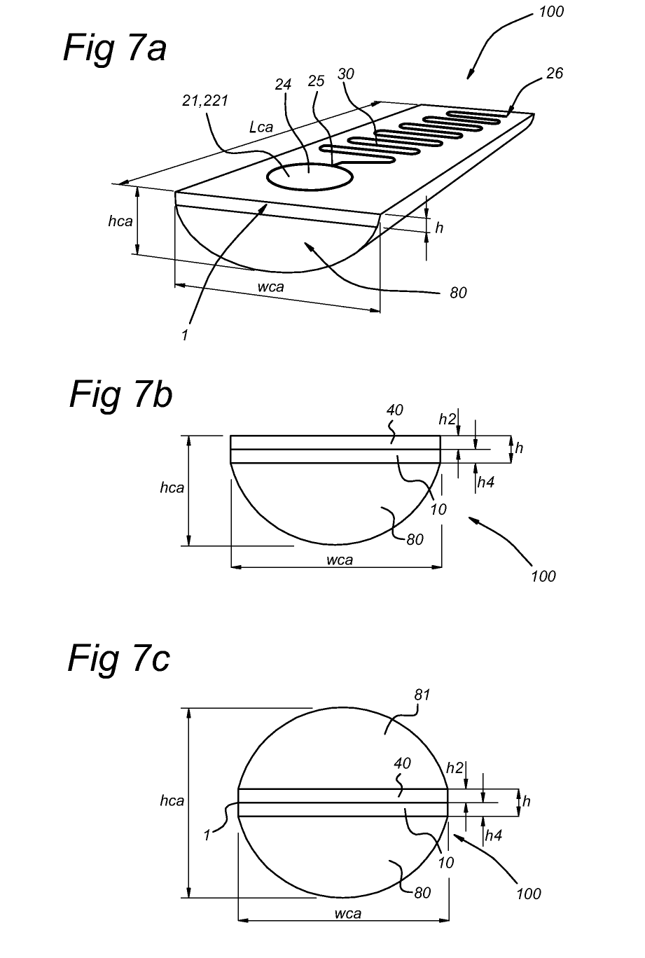

Device 1 may be designed such that an opening 21 is provided wherein the opening is directed to a side of device 1, i.e. channel 30 extends to an edge of body 10 and liquid may enter channel 30 at one end 25 of channel 30. Such an embodiment is shown in e.g. FIGS. 1 and 2. However, the device may also be designed such that opening 21 is provided to the top of device 1, i.e. cover 40 comprises an opening such that liquid may enter channel 30 via an opening in cover 40 into channel 30 at first end 25 (“top opening”). An example of such an embodiment is shown in FIG. 7a, with top opening 221. The one end 25 of channel 30, i.e. in fact opening 21, may comprise an opening compartment or broadening 24, such that entrance of GI liquid into channel 30 is facilitated. This broadening or opening compartment 24 may have a volume of about less than 1% of the total volume, e.g. 0.005-50 μl, in a preferred embodiment 0.05-5 μl. Preferably, when using a top opening 221, the body 10 comprises a corresponding opening, preferably of about the same diameter.

In a specific embodiment, there is provided a device wherein body 10 further comprises a reservoir 70 with a reservoir filling channel 72 and a connection channel 74 connecting reservoir 70 and channel 30. Reservoir 70 may be used to contain a quenching liquid or stabilizing liquid. While GI liquid travels from inlet or opening 21 in the direction of second end 26, GI liquid is contacted with liquid from reservoir 70. Hence, liquid from reservoir 70 will also travel in the direction of second end 26 induced by GI liquid transport in channel 30. Examples of quenching or stabilizing liquids are e.g. methanol or ethanol.

One may desire to tune the flow of GI liquid into channel 30 (i.e. sampling speed) or the flow of stabilizing liquid from reservoir 70 into channel 30. Tuning can be done by varying one or more parameters selected from e.g. the group consisting of channel length, width w1 and height h1 of channel 30, length, width and height of channel 74, dimensions like length, width and height of opening 21, pressure within channel 30 (for instance an initial pressure in channel 30 before sampling of 0.8 bar or smaller), etc., but one may also, or in addition to tuning one or more of these, include a kind of restriction or barrier in channel 30 or channel 74, respectively. In an embodiment, barrier 33 is provided in channel 30, just downstream of opening 21, and/or barrier 73 is located in connecting channel 74 (assuming a liquid in reservoir 70 travelling to channel 30 through connecting channel 74), downstream of the edge of reservoir 70. An example of such a restriction is shown in FIG. 4. The restrictions may e.g. have a height of 10-90% of the channel height, more preferably about 30-70% of the channel height (of channel 30 or 74, respectively), for example about 0.1-10 μm and a length of about 2-500 μm. The restrictions may e.g. be incorporated to control the time of filling. Restrictions 33 and 73 may be selected such bearing in mind the viscosity of the liquid(s) to be sampled, the viscosity of the quenching liquid, the period of time device 1 will sample, the mechanism by which it is sampled (by vacuum: pressure in channel; or by capillary forces: the strength of the capillary forces, or by a combination of vacuum and capillary forces). Restrictions 33 and 73 may be provided independently of each other. Restrictions or barriers may also be provided elsewhere in channel 30. Barriers or restrictions 33 and 73, or other barriers, may independently of each other also comprise hydrophobic barriers (see below).

Preferably, the mechanism by which the liquid is sampled, i.e. the driving force for sampling, is a vacuum within the channel structure. In this way, liquid is sucked into channel 30 at opening 21 at first end 25, and is drawn in the direction of second end 26. Thus, the liquid flow during sampling from the one end 25 in the direction of the second end 26 is preferably due to the presence of a vacuum in the channel structure. Hence, device 1 is preferably arranged to have a pressure lower than 1 bar in the channel structure during a substantial part of the sampling time. Optional side compartments 50 are filled with time, the compartments closer to first end 25 being filled with sampling liquid sampled earlier than the compartments closer to second end 26. Hence, in a preferred embodiment, channel 30 has during sampling only one opening 21 for sampling liquids; i.e. channel 30 has no second opening for instance at second end 26. During sampling, liquid only enters via opening or inlet 21 and due to the vacuum, liquid is sampled and the channel and optional side compartments fill with time. A second opening during sampling, which is open during sampling, would not allow the use of vacuum as driving force. Hence, as schematically depicted in the figures, device 1 according to the invention preferably has only one opening 21 for channel 30 arranged to sample liquids, such as GI liquids. Hence, during sampling, preferably a vacuum (pressure below 1 bar) in the channel structure is the driving force during a substantial part of the sampling time and liquid(s) to be sampled only enter channel 30 via opening or inlet 21 at first end 25. Therefore, the sampling device 1 preferably possesses one single opening 21 for sampling the liquid(s) (i.e. at first end 25). Thus, in a specific embodiment, device 1 is arranged to sample the liquid(s) by means of vacuum force when device 1 is in use for sampling the liquid(s).

At least part of body 10 comprises a material selected from the group consisting of silicon, glass, fused silica, quartz, ceramic and plastic. In a preferred embodiment, the material comprises silicon and body 10 is e.g. based on a Si wafer (see also below). In yet another preferred embodiment, the material of body 10 is a plastic, e.g. a thermoplastic polymer like those which can be derived from PMMA (polymethyl methacrylate), POM (polyoxymethylene), PC (polycarbonate), PCDF (polychlorinated dibenzofuran) and PSU (polysulfone), or other thermoplastic polymers known to the person skilled in the art such as e.g., but not limited to ABS (acrylonitrile butadiene styrene), PVC, (polyvinyl chloride), polypropylene, polyethylene, acrylic, celluloid, polystyrene, and cellulose acetate, or polymers like polydimethylsiloxane (PDMS).

The channel structure comprising channel 30, optional side compartments 50 (also indicated as compartments 50), etc., may be derived by providing these structures into body 10 (see also below). At least part of this structure is to be covered by cover 40 in order to provide a circumferentially closed channel 30 that can be used to collect GI liquid(s) during a certain period of time. The person skilled in the art will choose cover 40 such as to provide a suitable device 1 for sampling GI liquids and subsequently analysing the sampled GI liquids. Analysis can be performed in several ways (see below), and hence, different types of covers 40 may be provided.

In an embodiment, cover 40 comprises a plate substantially covering at least the whole area of channel 30. In a further embodiment, this may be a cover having the same length L and width w values as body 10. In yet another embodiment, cover 40 comprises a plate substantially covering at least the whole area of channel 30 and optional side compartments 50 (including side compartment channels); this may also be a cover having the same length L and width w values as body 10. Such cover 40 may in an embodiment have one opening as top opening 221 to first end 25 of channel 30. Hence, cover 40 is preferably arranged to cover channel 30 (from first end 25 (not including opening 21) to second end 26), optional side compartments 50 and optional side compartment channels 51.

In another embodiment, a cover 40 may be used having an inlet or opening 21 arranged such that liquid can flow through this opening 21 into channel 30 at first end 25, indicated as top opening 221 in FIG. 6.

In another embodiment, at least part of cover 40 is transparent for one or more of UV, VIS and IR radiation, i.e. the transmission under perpendicular irradiation of one or more of the UV, VIS or IR light is at least 20%, preferably at least 40% and more preferably at least 60%. The person skilled in the art understands that analysis with this radiation does not necessarily imply perpendicular irradiation of the top of device 1.

In yet another embodiment, cover 40 comprises one or more parts 45 with reduced height h3 at one or more positions above channel 30 and/or above optional side compartments 50 when arranged as cover 40 on body 10 (i.e. opposite to bottom 30b of channel 30 and/or opposite to bottom 50b of optional side compartment 50), cover 40 may have reduced height h3). This is schematically shown in FIG. 6, wherein part of cover 40 with height h2, has reduced height h3 (or cover width), opposite to bottom 30b of channel 30. A cover with height h2 and opposite of at least part of channel 30 reduced height h3 may advantageously provide a strong cover 40, but still at one or more places 45 above channel 30 penetrable for e.g. a needle of a syringe to extract sampled liquid, for further analysis. In general, height h2 of cover 40 is between about 0.5 μm and 2.5 mm, preferably between about 1 μm and 2.5 mm, more preferably between about 0.2 mm and 0.6 mm. Optional reduced height h3, may be about 10-80% of height h2, in an embodiment between about 0.5 μm and 2.5 mm, more preferably between about 1 μm and 1.2 mm. Referring to FIG. 6, height h of device 1 is the summation of height h4 of body 10 and height h2 of cover 40 (and including e.g. a glue 41 (if used), see e.g. FIG. 5).

Cover 40 may consist of one or more materials selected from the group consisting of silicon, glass, fused silica, quartz, ceramic, metal, rubber and plastic, but may amongst others also comprise polymers (for example those mentioned above for the body 10 material) or biocompatible materials known to the person skilled in the art. For example referring to FIG. 5, cover 40 may substantially comprise glass, further including a region with a filter material 22 arranged above or over opening 21, such as top opening 221, like a micro dialysis membrane (preferably molecular weight cut-off (MWCO) in the range of 1-500 kD (e.g. 10 kD) or micro sieves with pore sizes from about 0.1 μm to about 10 μm are used). Optionally, such filter material 22 may be supported by one or more supporting means 60. Such filter material 22 may also be applied when opening 21 is a side opening, as e.g. depicted in FIGS. 1 and 2.

Further, cover 40 may comprise one or more sample cover(s) 42, preferably selected and designed such that passage of gases (e.g. as described above) or fluids or preferably both gases and fluids are not allowed, arranged above channel 30 and/or optional side compartments 50, in order to enable extraction of liquid. For example, sample cover 42 may be a rubber, or may be a transparent material such as a plastic, selected to be transparent for radiation of a spectrophotometer (e.g. UV, VIS, IR excitation/emission, Raman, etc.) for analysis of the sampled liquid and/or selected to be able to be penetrated by a needle or syringe for sampling sampled GI liquid and analyzing the sampled liquid outside device 1, e.g. with MS (mass spectrometry), etc. Sample cover(s) 42, filter material 22 and other structures in cover 40 may be arranged in cavities within cover 40 and may be held, stitched, glued, fixed, or in any other way known to the person skilled in the art attached to cover 40 or body 10 or to both, e.g. as shown in FIG. 5 in an embodiment by a kind of fixation means 43. Cover 40 may be attached to body 10 by e.g. a glue, anodic bonding, etc., as indicated with reference number 41 in FIG. 5.

In yet another embodiment, cover 40 essentially consists of a glass wafer, in a further embodiment having a thickness h2 of about 10 μm-5 mm.

Further, the dimensions herein such as width, height, length may also vary through device 1. In a specific embodiment, width w1 of channel 30 (either plain channel or compartment embodiment, vide infra) may vary through at least part of channel 30. For example, width w1 may be smaller close to first end 25 and may increase towards second end 26 of channel 30. In an embodiment, the width may vary from 1 μm to 300 μm. Preferably, the ratio of the maximum width w1 and minimum width w1 is between 1 and 100, more preferably between 2 and 50. An advantage of an increasing width with increasing distance from first end 25 can be that the sampling rate may more easily be controlled. With a channel 30 having no varying width w1 of the channel, the sampling rate may decrease with filling (i.e. with sampling time), whereas selection of a ratio of the maximum width w1 (second end 26) and minimum width w1 (close to first end 25) larger than 1, the sampling rate may be substantially constant during 50% or more of the filing time of channel 30 (with optional side compartments 50).

According to an aspect of the invention, the body 10 comprising the channel structure described herein is provided. The extent of the protection of the claims therefore also includes a device 1 without cover 40 (bond to body 10).

Further, the person skilled in the art will understand that body 10 and cover 40 can be interchanged. Cover 40 can be considered as body 10, and vice versa. One may also provide a channel structure comprising channel 30 and optional side compartment(s) 50 in cover 40 or both in body 10 and cover 40. One channel structure may be partly present in body 10 and partly present in cover 40, forming one connected channel structure. Body 10 may also comprise more than one channel structure. Further, one may also use a number (≧2) of bodies arranged on top of each other, each body having one or more channel structures, which may be independent of each other (multiple sampling device).

“Plain” Channel

Referring to FIGS. 1 and 8a, during transport of device 1 through the GI tract, GI liquid enters through opening 21 into channel 30 and travels in the direction of second end 26. In this “plain” embodiment, after sampling, a “sample” taken at to (starting time of sampling liquid(s)) will be found closest to second end 26 (i.e. the liquid front within channel 30), whereas a “sample” taken at tend (last moment of sampling) will be found closest to end 25. For example, GI liquid that has been sampled and has filled channel 30 up to a position 96, reflects at position 96 liquid sampled at to, whereas liquid just in the beginning of channel 30, indicated with reference number 95, is liquid sampled at tend (or substantially tend). As will be clear to the person skilled in the art, since device 1 samples continuously, the sampled liquid within channel 30 will not comprise “discrete” samples, but provides a type of continuous sampling wherein the presence of species contained in the sampling liquid closer to end 26 reflects the presence of species in the GI channel where device 1 starts sampling (i.e. to), and the presence of species contained in the sampling liquid closer to end 25 reflects the presence of species in the GI channel where device 1 ends sampling (i.e. tend, or substantially tend), and all intermediate samples (which may vary through the GI tract and hence also through channel 30 after sampling) reflect “samples” taken at intermediate times, i.e. at intermediate positions within the GI tract (i.e. intermediate times during the total time device 1 is sampling in the GI tract until channel 30 is full). Likewise, if certain species are present, the concentration thereof contained in the sampling liquid closer to end 26 reflects the concentrations of species in the GI channel where device 1 starts sampling (i.e. to), and the concentration of species contained in the sampling liquid closer to end 25 reflects the concentrations of species in the GI channel where device 1 ends sampling (i.e. tend, or substantially tend), and all intermediate concentrations (which may vary through the GI tract and hence also through channel 30 after sampling) reflect “samples” taken at intermediate times, i.e. at intermediate positions within the GI tract (i.e. intermediate times during the total time device 1 is sampling until channel 30 is full or device 1 being removed from the liquid to be sampled). As mentioned above, the driving force for sampling is preferably a vacuum in the channel structure.

Preferably, the total volume of channel 30 in the “plain” embodiment schematically depicted in FIG. 1 is selected such that a number of samples can be taken. Samples of about 10 nl, preferably about 20 nl, more preferably about 50 nl may suffice for analysis of e.g. acetate, propionate, butyrate, lactate, formate, ammonia, amino acids, sugars, etc. by processes such as GC-MS, LC-MS, FT-MS. In a preferred embodiment, the total volume of channel 30 is about 0.2 μl-4000 μl, more preferably about 0.5 μl-3500 μl, even more preferably about 1.5 μl-3000 μl. Herein, the total volume is the volume of channel 30 from first end 25 at inlet 21 to second 26 at the end of channel 30.

In an embodiment, the depths of channel 30, and optional reservoir 70 and channel 74 can be substantially different.

Device 1 according to the invention comprises a preferred embodiment, a channel 30 having a length of about 0.1-25 m, more preferably about 1-10 m. Device 1 according to the invention comprises in another preferred embodiment a channel 30 having a width w1 of about 2-300 μm, more preferably about 10-50 μm. Device 1 according to the invention comprises in yet another preferred embodiment a channel 30 having and a depth h1 of about 2-4000 μm, more preferably about 100-300 μm.

Hence, in a specific embodiment device 1 comprises a channel having a length of 0.1-25 m, a width of 2-300 μm and a depth of 2-4000 μm. In a more specific embodiment device 1 comprises a channel 30 having a length of 1-10 m, a width w1 of 10-50 μm and a depth h1 of 100-300 μm.

In the table below, some embodiments are summarized:

TABLE 2
Example of dimensions for a number of
“plain channel” embodiments

| | | | | | Minimum | | Maximum | |
|---|---|---|---|---|---|---|---|---|
| | | | | | value of | | value of | |
| | Minimum | | Maximum | | preferred | | preferred | |
| | value of an | | value of an | | embodi- | | embodi- | |
| | embodiment | | embodiment | | ments¹ | | ments² | |
| Length | 0.1 | m | 25 | m | 1 | m | 10 | m |
| channel | | | | | | | | |
| of “plain” | | | | | | | | |
| channel | | | | | | | | |
| Width w1 | 2 | μm | 300 | μm | 10 | μm | 50 | μm |
| channel | | | | | | | | |
| of “plain” | | | | | | | | |
| channel | | | | | | | | |
| Height h1 | 2 | μm | 4000 | μm | 100 | μm | 300 | μm |
| channel | | | | | | | | |
| of “plain” | | | | | | | | |
| channel | | | | | | | | |
| Volume | 5 | nl | 4500 | μl | 0.2 | μl | 4000 | μl |
| channel | | | | | | | | |
| of “plain” | | | | | | | | |
| channel | | | | | | | | |

¹other (more) preferred embodiments may have larger minimum values
²other (more) preferred embodiments may have smaller maximum values

Compartment Device

Channel 30 may optionally further comprise one or more side compartments 50, as schematically depicted in FIGS. 2 and 8b. Channel 30 of device 1 with one or more side compartments 50, has a total volume of about 5 nl-4500 μl. Preferably, the total volume of channel 30 and side compartments 50 is about 0.2 μl-4000 μl, more preferably about 0.5 μl-3500 μl, even more preferably about 1.5 μl-3000 μl. Herein, the total volume is the volume of channel 30 from first end 25 at inlet 21 to second 26 end at the end of channel 30, including the volume of side compartments 50 and if present, side channels 51. Channels 72 and 74 and reservoir 70 do not contribute to this total volume. Side channels 51 may have a length of about 0-5000 μm, preferably about 300-1000 μm. The width of side channel(s) 51 may be about 1-2000 μm, preferably about 30-200 μm; the height of the side channel(s) 51 may be about 0.2-4000 μm, preferably 0.2-10 μm, more preferably about 0.5-5 μm. The height of side channel 51 is preferably the same or substantially the same as the height h1 of channel 30.

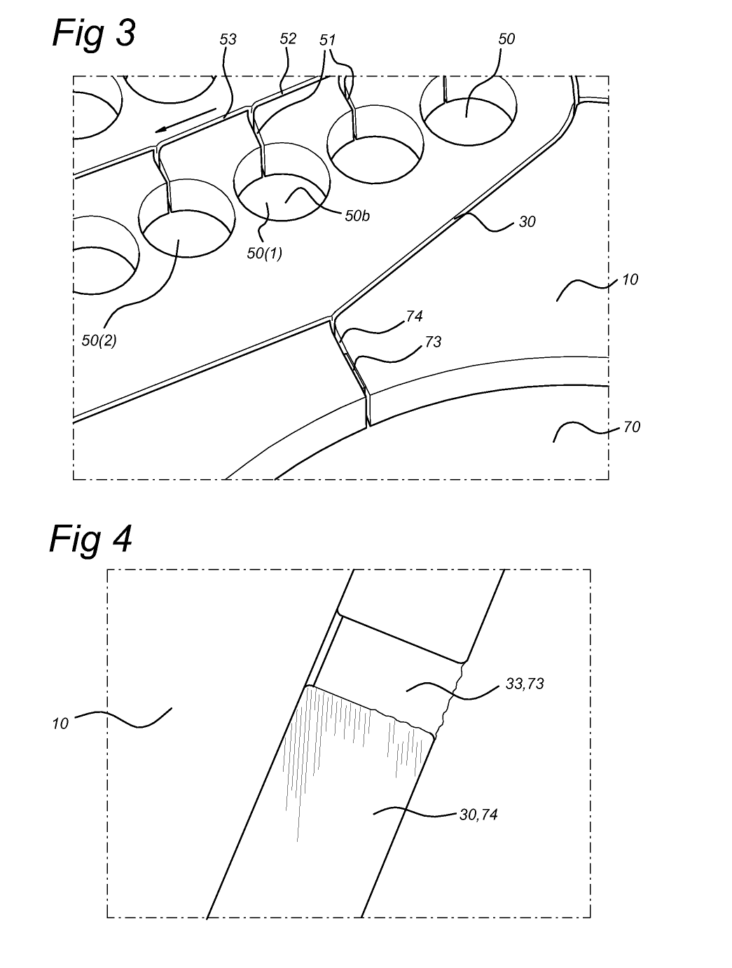

Side compartments 50 may be directly adjacent to channel 30 (i.e. no compartment channel 51), for instance such that compartments 50 are (bulges) arranged in channel 30, but may also be more remote from channel 50, being in liquid contact with channel 30 via compartment channel 51. A detail of an embodiment of device 1 having side compartments 50 which are connected by side channel 51 to channel 30 is depicted in FIG. 3. In this embodiment, side channels 51 are slightly curved channels, having a smaller width close to side compartment 50 and a larger width close to channel 30. Further, the curvature is such that it is convex with respect to the flow of GI liquid into channel 30 when sampling (the flow direction in FIG. 3 is from side compartment 50(1) to side compartment 50(2), indicated with an arrow). Relative to side compartment 50(1), reference number 52 refers to the upstream part of channel 30 and reference number 53 refers to the downstream part of channel 30, as can be derived from FIG. 3. An advantage of the curvature and narrowing of side channel 51 as depicted in FIG. 3 is that GI liquid relatively easily enters compartment 50 and backflow into channel 30 is minimised. In this way, sampled liquid may be contained during sampling, avoiding unintended mixing of the sampled liquid in a compartment 50 with travelling liquid in channel 30.

In an embodiment, the depths of channel 30, and optional reservoir 70 and channel 74 can be substantially different. For example, channel 30 may have a depth h1 of about 0.2-10 μm (for instance 0.5-5 μm) and reservoir 70 may have a depth of 100-300 μm. In an embodiment, the depths of channel 30, optional side channel 51, and side compartment(s) 50 can be substantially different. For instance, depth/height h1 of channel 30 and side channel 51 may be about 0.2-10 μm, preferably 0.5-5 μm and depth dc (see FIG. 5) of side compartment 50 may be about 100-300 μm.

In yet another embodiment, containing of GI liquid in side compartment(s) 50 may be promoted by providing a type of hydrophobic barrier behind one or more compartment(s) 50 (reference number 50(1) in FIGS. 2 and 3) in channel 30, just after side channel 51 and before a downstream compartment 50 (reference number 50(2) in FIGS. 2 and 3), e.g. as indicated with reference number 53. Such hydrophobic barrier may be applied by depositing a coating in channel 30. Such a coating may provide the effect that GI liquid relatively easily enters compartment 50 (through optional side channel 51), but only with difficulty can flow further in channel 30 until compartment 50 is completely filled. An example of a hydrophobic coating is for instance a polyfluoroalkyl coating using a polyfluoroalkyltrichlorosilane. This reagent binds (covalently) to a glass or silicon-oxide surface and the coating even withstands temperatures up to 400° C. In an embodiment, the surface may be treated for 5 minutes in a 1H,1H,2H,2H-Perfluorodecyltrichlorosilane/iso-Octane solution (1%) under a nitrogen atmosphere. For instance, the surface of channel 50 (i.e. the bottom and side walls of channel 50) are treated at position 53, just behind a side channel 51 (for example 1-100 μm downstream relative to an opening in channel 30 to side compartment 50 (like side channel 51) over a length of 1-200 μm. The coating thickness may be a monolayer, e.g. about 0.5-3 nm). Preferably, the hydrophobic barrier, such as a coating, is selected to provide a contact angle with water of at least 90°, more preferably at least 130°, yet more preferably at least 150°, yet even more preferably at least 160°, even more preferably at least about 170°, such as at least 180°. Below, another embodiment of the hydrophobic barrier is described (“black substrate”).

The integration of hydrophobic barriers is especially suitable for sampling devices 1 which are designed to sample hydrophilic liquids. However, in another embodiment, instead of hydrophobic barriers, hydrophilic barriers are integrated in the sampling device. This may for instance be of interest for sampling devices 1 which are designed to sample hydrophobic liquids such as oil, etc.

Referring to FIG. 2, during transport of device 1 through the GI tract, GI liquid enters through opening 21 into channel 30 and travels in the direction of second end 26. After sampling, a “sample” taken at to will be found in a compartment 50 closest to first end 25 (i.e. closest to opening 21), whereas a “sample” taken at tend will be found in a compartment 50 closer to end 26. The compartment embodiment may therefore provide samples in compartments 50 which are more discrete. Referring to FIGS. 2 and 3, for example, a compartment 50(1) comprises species contained in the sampling liquid sampled during a certain first time frame, showing the presence of these species in the GI tract sampled during this certain first time frame (i.e. a certain first distance device 1 has travelled in the GI tract), and a next compartment 50(2) contains a sample comprising species representing GI liquid sampled during a next time frame (i.e. sampled during a next part of the GI tract traversed after the first time frame). Hence, the presence of species in a compartment 50(1) closer to end 25 and opening 21 shows the presence of species in the GI channel where device 1 starts sampling (see also below) and the concentration of species contained in the sampling liquid closer to end 26 shows species in the GI channel where device 1 ends sampling and all intermediate samples (which may vary through the GI tract and hence also through channel 30 after sampling) show the presence of species taken at intermediate times, i.e. at intermediate positions within the GI tract. Hence, if certain specific species are present, a compartment 50(1) will have concentration(s) of species contained in the sampling liquid sampled during a certain first time frame, representing the concentration(s) of this/these species in the GI tract “sample” sampled during this certain first time frame (i.e. a certain first distance device 1 has travelled in the GI tract), and a next compartment 50(2) contains a “sample” having concentration(s) of species representing GI liquid sampled during a next time frame (i.e. sampled during a next part of the GI tract traversed after the first time frame. Hence, concentrations of species in a compartment 50 closer to end 25 and opening 21 reflects the concentrations of species in for example the GI channel where device 1 starts sampling (see also below) and the concentration of species contained in the sampling liquid closer to end 26 reflects the concentrations of species in the GI channel where device 1 ends sampling and all intermediate concentrations (which may vary through the GI tract and hence also through channel 30 after sampling) reflect “samples” taken at intermediate times, i.e. at intermediate positions within the GI tract. Likewise, this applies to sampling in other systems than the GI tract.

Likewise, the total volume of the compartments 50 in channel 30 in the “compartment” embodiment schematically depicted in FIG. 2 is selected such that a number of samples can be taken. Hence, in a preferred embodiment, the total volume of compartments 50 is at least about 0.1 μl or more (e.g. 5 compartments 50 of 20 nl, or 2 compartments 50 of 50 nl), more preferably at least about 0.2 μl or more (e.g. 10 compartments 50 of 20 nl, or 4 compartments 50 of 50 nl) even more preferably about 0.5 μl or more (e.g. 25 compartments 50 of 20 nl, or 10 compartments 50 of 50 nl), yet even more preferably about 1.5 μl or more (e.g. 75 compartments 50 of 20 nl, or 30 compartments 50 of 50 nl), even more preferred about 2.5 μl or more (e.g. 125 compartments 50 of 20 nl, or 50 compartments 50 of 50 nl). In an embodiment, device 1 comprises at least 2 or more, preferably at least 10 or more side compartments 50. In other embodiments, device 1 comprises more preferably at least 20, even more preferably at least 50 and even more preferably at least 100 compartments 50.

In an embodiment, each side compartment 50 comprises a volume of about 2 nl-1.5 μl. Preferably, each side compartment 50 substantially comprises the same volume, i.e. volume variations within 5%. The volume of the side compartments 50 is selected such that species to be determined are in a sufficient quantity present to be detected by a predetermined detection method in at least one of side compartments 50.

In a specific embodiment, device 1 according to the invention comprises side compartments 50, each having a volume of about 2 nl-1.5 μl, more preferably about 10-1000 nl, and channel 30 having a length of about 0.002-0.5 m, more preferably about 0.01-0.2 m. In another specific embodiment, device 1 according to the invention comprises side compartments 50, each having a volume of about 2 nl-1.5 μl, more preferably about 10-1000 nl, and channel 30 having a width w1 of about 2-300 μm, more preferably about 10-100 μm. In yet another specific embodiment, device 1 according to the invention comprises side compartments 50, each having a volume of about 2 nl-1.5 μl, more preferably about 10-1000 nl, and channel 30 having a depth h1 of 0.2-4000 μm, more preferably about 1-5 μm.

In a preferred embodiment, device 1 comprises channel 30 having a length of 0.002-0.5 m, a width w1 of 1-300 μm and a depth h1 of 0.2-4000 μm, further comprising side compartments 50 each having a volume of 2 nl-1.5 μl. In a preferred embodiment, device 1 comprises a channel 30 having a length of 0.01-0.2 m, a width of 10-50 μm and a depth h1 of 0.2-10 μm (preferably 0.5-5 μm), further comprising side compartments 50 each having a volume of 10-1000 nl.

In an embodiment, compartment(s) 50 have a height/depth dc of about 10-4000 μm, more preferably about 100-300 μm. In yet another embodiment, compartment(s) 50 have width or diameter wdc of about 100-2000 μm, more preferably about 300-1000 μm. In a preferred embodiment, the width of the channel is about 50-150 μm (such as 150 μm) and the height of the channel is about 0.2-10 μm (such as 4 μm); the depth of the compartments is about 50-150 μm (such as 150 μm), the diameter of the compartments is about 500-1500 μm (such as 800 μm), and the device 1 has about 20-100 compartments (such as 45. All embodiments herein depict compartments 50 having a circular shape. However, compartments 50 may also be rectangular or cubic. Preferably, the length and width of such compartments 50 are about 100-2000 μm, more preferably about 300-1000 μm. Further, side compartments 50 may be present at one or at both sides of channel 30.

In the table below, some embodiments are summarized:

TABLE 3
Example of dimensions for a number of
“compartment” embodiments

| | Min. | | Max. | | Min. | | Max. | |
|---|---|---|---|---|---|---|---|---|
| | value | | value | | value of | | value of | |
| | of an | | of an | | preferred | | preferred | |
| | embodi- | | embodi- | | embodi- | | embodi- | |
| | ment | | ment | | ments¹ | | ments² | |
| Length | 0.002 | m | 0.5 | m | 0.01 | m | 0.2 | m |
| channel of | | | | | | | | |
| “compartment | | | | | | | | |
| embodiment” | | | | | | | | |
| Width w1 | 1 | μm | 300 | μm | 10 | μm | 100 | μm |
| channel of | | | | | | | | |
| “compartment | | | | | | | | |
| embodiment” | | | | | | | | |
| Height h1 | 0.2 | μm | 4000 | μm | 0.5 | μm | 5 | μm |
| channel of | | | | | | | | |
| “compartment | | | | | | | | |
| embodiment” | | | | | | | | |
| Depth dc | 10 | μm | 4000 | μm | 100 | μm | 300 | μm |
| of compartment | | | | | | | | |
| 50 of | | | | | | | | |
| “compartment | | | | | | | | |
| embodiment” | | | | | | | | |
| Width/diameter | 100 | | 2000 | | 300 | | 1000 | |
| wdc of | | | | | | | | |
| compartment | | | | | | | | |
| 50 of | | | | | | | | |
| “compartment | | | | | | | | |
| embodiment” | | | | | | | | |
| Volume | 2 | nl | 1.5 | μl | 10 | nl | 1000 | nl |
| of side | | | | | | | | |
| compartment | | | | | | | | |
| 50 of | | | | | | | | |
| “compartment | | | | | | | | |
| embodiment” | | | | | | | | |
| Total | | | | | 0.1 | μl | ≧2.5 | μl |
| volume | | | | | | | | |
| of side | | | | | | | | |
| compartments | | | | | | | | |
| 50 of | | | | | | | | |
| “compartment | | | | | | | | |
| embodiment” | | | | | | | | |
| Total | 5 | nl | 4500 | μl | 0.2 | μl | 4000 | μl |
| volume of | | | | | | | | |
| channel 30, | | | | | | | | |
| side | | | | | | | | |
| compartments | | | | | | | | |
| 50 (and | | | | | | | | |
| optional | | | | | | | | |
| side channels | | | | | | | | |
| 51) of | | | | | | | | |
| “compartment | | | | | | | | |
| embodiment” | | | | | | | | |

¹other (more) preferred embodiments may have larger minimum values
²other (more) preferred embodiments may have smaller maximum values

In an embodiment, cover 40 of device 1 with channel 30 with a number of side compartments 50 comprises one or more parts 45 with reduced thickness h3 at one or more positions in cover 40 where the parts with reduced thickness h3 are opposite to the one or more side compartments 50 and/or at least part of channel 30 (when cover 40 is arranged on body 10). For example referring to FIG. 5, above each side compartment 50, a sample cover 42 arranged above channel 30, in order to enable extraction of liquid after sampling GI liquids. Such sample cover 42 may be a rubber, as described above, substantially not enabling transport of GI liquids through such sample cover 42, but in an embodiment penetrable by a syringe needle, in another embodiment transmissive for radiation, e.g. UV, VIS or IR, and in an yet another embodiment both penetrable by a needle and transmissive for radiation. Parts with reduced thickness may also be present above/opposite to at least part of a channel 30. In this context, opposite refers to the position opposite of the bottom surface of channel 30 or side compartment 50. For example in FIG. 3 a bottom surface 50b of compartment 50(1) is shown and in FIG. 6 a bottom surface 30b of channel 30 is shown, and opposite of this bottom surface 50b and/or 30b, respectively, at least part of cover 40 may comprise reduced thickness 45.

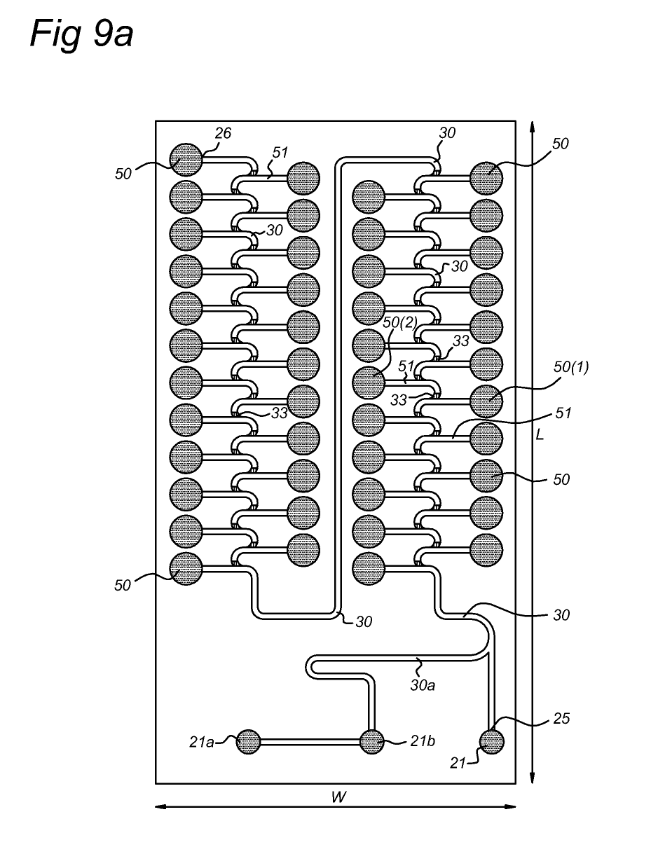

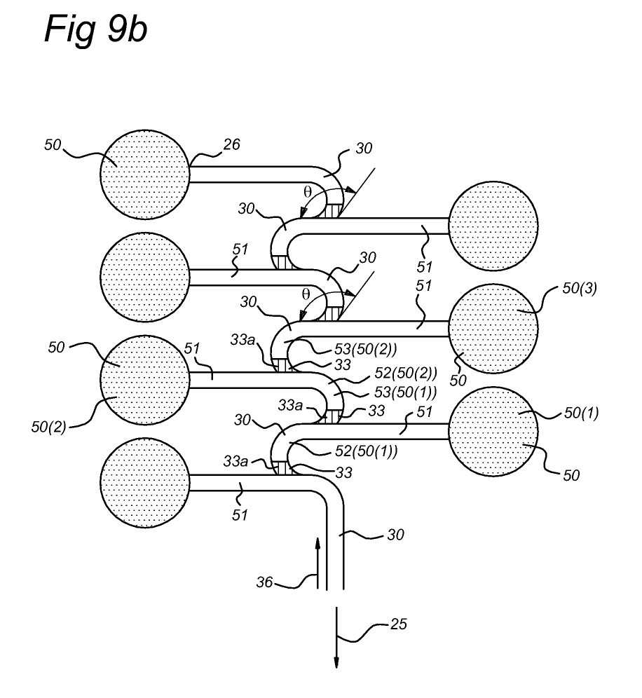

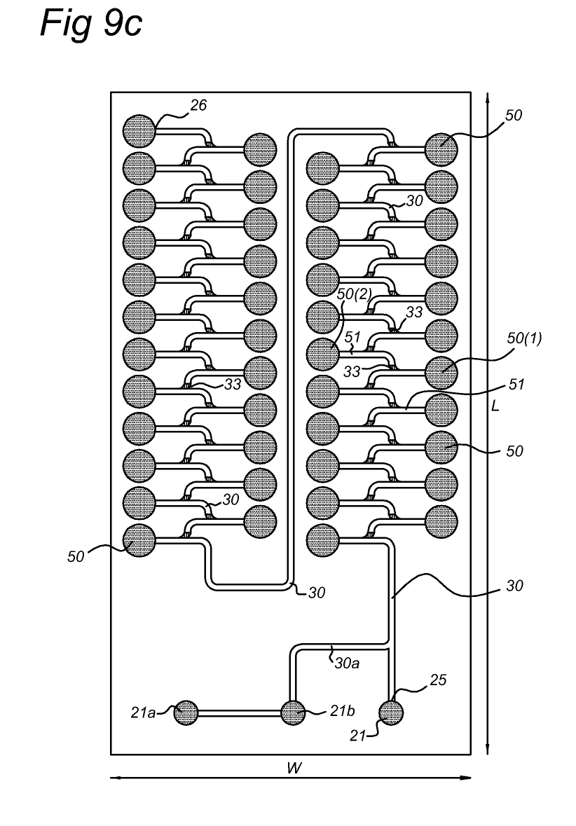

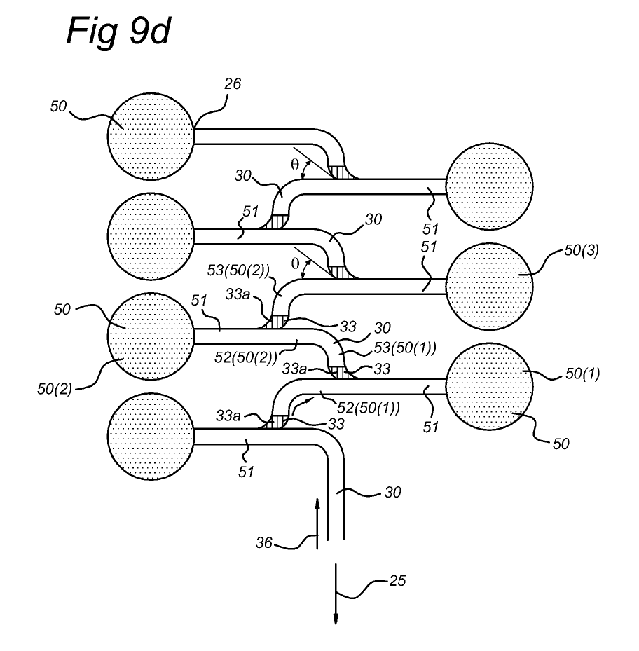

FIGS. 9a and 9c schematically show other preferred embodiments of device 1 according to the invention; FIGS. 9b and 9d schematically show enlargements of FIGS. 9a and 9c, respectively. Referring to FIG. 9a, a channel structure is depicted with channel 30, having an opening 21 at first end 25. For instance, due to a vacuum in the channel structure, liquid will flow from first end 25 in the direction of second end 26 (this is indicated with arrow 36). Second end 26 may be at a “last” compartment or may be a “dead end” of channel 30 (i.e. an extension of channel 30 beyond a last bifurcation to a last side compartment channel 51 (see also FIGS. 1 and 2)). Channel 30 shows a specific type of meandering structure. FIG. 9a (and 9b) shows one type, FIG. 9c (and 9d) shows another type. Channel 30 (which is the main channel) is in fact a branch of each side channel 51. Hence, the curvatures of the meandering structure are preferably such that (GI) liquid relatively easily enters side compartment 50 and backflow into channel 30 is minimised. In this way, sampled liquid may be contained in the side compartments 50 during sampling, substantially avoiding unintended mixing of the sampled liquid in the side compartments 50 with liquid travelling in channel 30. Nevertheless, during sampling, channel 30 may quickly be filled with a film of liquid, which appears however not to substantially influence the containment of the liquid samples in the side compartment 50 during sampling by the device 1 (see also below).

The device 1 comprises a plurality of compartments 50, for instance at least 10, more preferably at least 20 side compartments 50. The side compartments 50, of the plurality of side compartments 50 are connected to channel 30 via side compartment channels 51. Relative to all, or at least part of all side compartments 50, channel 30 comprises an upstream part 52 and a downstream part 53, relative to the side compartment channels 51. This is indicated in more detail in FIG. 9b. For instance, assuming first, second and third compartments 50, indicated with reference numbers 50(1), 50(2) and 50(3) respectively, reference number 52 (50(1)) refers to the upstream part of channel 30, relative to side compartment 50(1) and reference number 53 (50(1)) refers to the downstream part 53 of channel 30 relative to side compartment 50(1). However, this part of channel 30 is also an upstream part 52 relative to side compartment 50(2), as indicated with reference number 52 (50(2)). As can be seen in FIGS. 9a and 9b, the side compartment channels 51 are substantially in line with the flow direction of the liquid in the upstream part of channel 30, i.e. in this embodiment, side compartment channels 51 are substantially a prolongation or continuation of the upstream part 52 of channel 30. Channel 30 is arranged to “bend away” from all, or at least part of all side compartment channels 51. In this way, liquid will flow through channel 30 and will substantially fill side compartment 51(1) first. Having substantially filled this compartment 50(1), side compartment channel 51 is substantially filled with liquid, and due to the pressure of the liquid, liquid will find its way to the next compartment and flow into the downstream part 53 (i.e. 53 (50(1))) of channel 30. Compartment 50 after compartment 50 is filled in this way, until sampling stops or the sampling liquid has reached end 26 of channel 30. This end may be at a “last” compartment 50 or may be a dead end.

Hence, in a preferred embodiment device 1 is provided wherein this sampling device 1 is arranged to have a liquid flow 36 from the first end 25 of the channel 30 in the direction of second end 26 of channel 30, wherein channel 30 comprises a plurality of side compartments 50, wherein the compartments 50 of the plurality of side compartments 50 are connected to channel 30 via side compartment channels 51, wherein channel 30 comprises upstream part 52 and downstream part 53 relative to the side compartment channels 51, respectively, and wherein side compartment channels 51 are arranged to be substantially in line with the flow direction 36 in the upstream parts 52 of channel 30, respectively. Note that in the embodiment schematically depicted in FIGS. 9a and 9b, side compartment channel 51 and channel 30 may be considered to coincide for a last compartment 50.

The fact that the side compartment containing channel structures depicted herein comprise side compartments 50 at both sides of channel 30 does not exclude embodiments wherein the side compartments are arranged at only one side of channel 30 or other arrangements of the side compartments 50 (see also FIG. 2).

FIGS. 9c and 9d schematically depict a similar embodiment as depicted in FIGS. 9a and 9b. The main difference is that the branch of channel 30, bending away from side compartment channel 51 bends away in another direction. In general, any angle θ larger than 0° and smaller than 180° can be selected. In FIG. 9a, the angle θ wherein channel bends away from side compartment channel 51 is between about 20 and 80°; in FIGS. 9c and 9d, this angle θ is between about 110° and 170°.

The preferential filling of a side compartment 50 above the filling of down stream part of channel 30 (relative to the side compartment channel 51 to that compartment 50) can further be promoted by providing a barrier 33, preferably a hydrophobic barrier, downstream of the bifurcation in side compartment channel 51 and channel 30 (i.e. downstream part relative to this side compartment channel). Preferably, such a barrier is arranged just after the bifurcation, preferably within 10 μm from the bifurcation, but preferably in a distance after the bifurcation as small as possible, in order to prevent a large dead volume upstream in channel 30. In FIGS. 9a-9d, the hydrophobic barriers are indicated with reference number 33a. Hence, in a specific preferred embodiment, the hydrophobic barriers 33a are arranged to favour the flow in the direction of the side compartment 50. Preferably, the hydrophobic barrier 33a comprises a material or a structure such that the hydrophobic barrier 33a has a contact angle for water of at least 130°, more preferably at least about 170°. Preferably, the hydrophobic barriers 33a comprise black silicon (see also below).

Hence, in an embodiment channel 30 comprises a plurality of side compartments 50, wherein compartments 50 of the plurality of side compartments 50 are connected to the channel 30 via side compartment channels 51, and wherein in channel 30 hydrophobic barriers 33a are arranged to favour the flow in the direction of side compartment 50. Preferably, such hydrophobic barriers 33a are arranged in channel 30 downstream of each bifurcation into channel 30 and side compartment channel 51, as indicated in FIGS. 9a-9d (preferably within 10 μm from the bifurcation).

Referring to FIGS. 9a-d, in a preferred embodiment, device 1 is provided, wherein device 1 comprises first compartment 50(1) and channel 30 comprises upstream part 52(50(1) and downstream part 53(50(1) relative to side compartment channel 51 to the first side compartment 50(1). Further, device 1 further comprises second compartment 50(2) and channel 30 comprises upstream part 52(50(2) and downstream part 53(50(2) relative to side compartment channel 51 to second side compartment 50(2).

In general, device 1 will comprise a number of such first and second side compartments 50(1) and 50(2) respectively. As will be clear to the person skilled in the art, these reference symbols are only used to discriminate between substantially similar side compartments. For instance referring to FIGS. 9b and 9d, 7 side compartments 50 are depicted. The term, a “first side compartment” also includes a number of such side compartments. Preferably, device 1 according to the invention comprises at least 10 of such side compartments 50. Each upstream side compartment can be indicated as first compartment and each down stream compartment can be indicated as second compartment. Hence, as will be clear to the person in the art, an upstream side compartment (or first compartment) can also be a downstream side compartment (or second compartment), as it is the case for side compartment 50(2) in FIGS. 9b and 9d, which are down stream compartments relative to side compartments 50(1) in which are upstream compartments relative to side compartments 50(3).

Hence, relative to liquid flow 36 in channel 30 from the first end 25 in the direction of the second end 26, first side compartment 50(1) is arranged upstream relative to the second side compartment 50(2). Preferably, channel 30 is designed to have a flow resistance for the liquid to flow from upstream part 52(50(1) into the side compartment channel 51 to first side compartment 50(1) which is substantially smaller than a flow resistance for the liquid to flow from the upstream part 52(50(1) into the upstream part 52(50(2) of channel 30.

As will be clear to the person skilled in the art, when sampling hydrophobic liquids, barriers 33 may preferably comprise hydrophilic barriers. Hence, in an embodiment channel 30 comprises a plurality of side compartments 50, wherein compartments 50 of the plurality of side compartments 50 are connected to the channel 30 via side compartment channels 51, and wherein in channel 30 hydrophilic barriers (which may also be indicated with reference 33a) are arranged to favour the flow (of the hydrophobic liquid(s) which are sampled) in the direction of side compartment 50.

Device of the invention may be obtained by different production methods. In an aspect of the invention, there is provided a process for the production of a sampling device 1 for sampling liquid(s), especially for sampling liquid(s) from the gastrointestinal tract comprising a) providing a channel 30 and an opening 21 for entrance of the liquid(s) at one end 25 of channel 30 in a body 10 and optionally providing one or more side compartments 50 to channel 30, channel 30 having a length of 2 mm-25 m and a perimeter of 2.4-8600 μm, and channel 30 with the optional one or more side compartments 50 having a volume of 5 nl-4500 μl, and b) binding a cover 40 to at least part of body 10 such that channel 30 of body 10 is at least partially covered by cover 40.

As mentioned above, at least part of body 10 comprise a material selected from the group consisting of silicon, glass, fused silica, quartz, ceramic and plastic.

For example, silicon, can be etched to obtain the channel structure according to the invention with techniques known in the art like wet etching and dry etching. Lithographic techniques known in the art may be applied to a silicon wafer, using a mask designed to provide the desired pattern on the silicon wafer by the lithographic technique, thereby providing one or more bodies with a channel structure, and then sawing the body from the wafer.

For example, the channel structure according to the invention can also be obtained in glass, fused silica, quartz, and ceramic by wet or dry chemical etching techniques (known by the person skilled in the art).

In plastic, the structure can be provided by different methods e.g. LIGA or hot embossing. Hence, according to an embodiment of the invention, there is provided a process wherein channel 30 and optional one or more side compartments 50 (and other features such as the optional side channels 31, etc.) are provided in body 10 by a process selected from the group consisting of an etching process, a hot embossing process and a LIGA process.

LIGA is a well known technique. The word “LIGA” is an acronym from German words for lithography, electroplating, and moulding. As originally implemented, highly parallel x-rays from a synchrotron are incident on a mask patterned with high Z absorbers. The absorbers on the mask are thick enough to prevent the penetration of x-rays. In the open areas of the mask, the radiation passes through and exposes e.g. PMMA (polymethylmethacrylate) resist. The resist is then developed and the resulting PMMA mould is used to produce a metal part by electroplating in the developed regions. The electroplating is either the final step in the process or the electroplated part is used as a mould for replication from another material such as plastic or ceramic. Preferably, when applying LIGA in the method of the invention, an electroplated mould is used.

Another useful method is hot embossing. In a hot embossing process, a micro structured die (mould insert) having a mirror image of the channel structure of the invention is pressed into a thermoplastic polymer film under great force, the film having been heated beyond its glass transition temperature. The polymer fills the mould insert, in this way creating a detailed image of the microstructure of the channel structure. Subsequently, the film is cooled, and the replicated structure is released from the mould insert.

Hydrophobic barriers, such as hydrophobic barriers 33a indicate above may comprise “black silicon”, or more in general, since body 10 may also comprise other materials than silicon, may comprise “black substrate”. The terms “black silicon” or “black substrate” are for instance described in H. Jansen et al., J. Micromech. Microeng. 5 (1995) 115-120, which is incorporated herein by reference. It refers to substrate material that has undergone an etching process such that a kind of “grass” structure is obtained, that substantially absorbs all visible light.

The formulation of the black silicon method can be summarized by:

(1) Place a piece of silicon in the reactor and adjust the preferred power and pressure for an SF₆/O₂ plasma. Etch ca. 1 μm of silicon, open the process chamber, and inspect to ascertain if the silicon has turned black. If not, repeat the procedure but increase the oxygen flow. Proceed with this sequence until the wafer is black. Increasing the oxygen too much will still give rise to black-, or better grey-, silicon as there exists a positive tapered profile substantially without any underetching. Alternatively, it is possible to sense the black silicon with the help of a laser/photodetector set-up. Note, be sure that the thermal contact of the sample with the e.g. water cooled powered electrode is properly controlled, use vacuum grease, mechanical clamping, helium backside cooling, and so forth.

(2) After the black silicon regime is satisfactory found, add some CHF₃, to the mixture and increase this flow until the wafer is clean again. Too much CHF₃, will make the profiles isotropic (and smooth) because the CFₓ species scavenge the oxygen radicals required for the blocking layer.

(3) Now a wafer with the mask pattern of interest is inserted in the reactor and the etched profile is checked. Increasing the SF₆, content will create a isotropic profile. Adding too much oxygen will make the profile positively tapered and extra CHF₃ will make it more negatively tapered. Adding at the same O₂ and CHF₃ with the correct balance will create very smooth and nearly vertical walls. Increasing the pressure or decreasing the power will make the profile more positively tapered. Increasing at the same time the O₂ and CHF₃, flow, pressure and CHF₃, flow, power and O₂ flow, or power will hardly change the profile. However, such an increase will increase the DC self-bias and a higher DC self-bias will give the off-normal ions the energy to etch the side walls, thus changing the profile a little. Structure heights of 100 μm with an undercut of less than 1 μm are achieved.

A similar method may be applied for non-Si substrates (“black substrate”). Preferably, Si substrates are used for providing body 10 with the channel structure.

An alternative method as described above is described in example 7.

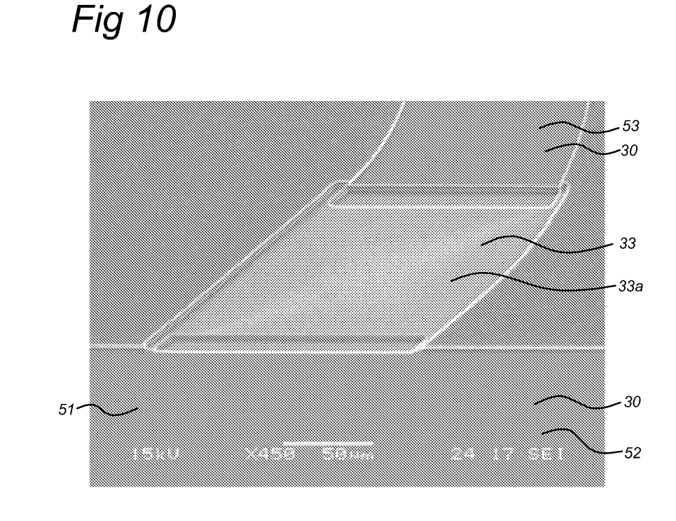

The barriers 33a may preferably be obtained by etching the channel structure and then etching the hydrophobic barriers 33a in the channel structure. However, it is also possible to etch the channel structure while masking the positions of the hydrophobic barriers 33a to be, and in the second etching process etch the hydrophobic barriers 33a into the channel 30. The former method has been applied for the hydrophobic barrier of FIG. 10. Due to possible overlap, a small part of channel 30 may be etched away twice. This is the case in FIG. 10, wherein two deepenings are found, one at the bifurcation of channel 30 (upstream) and side compartment channel 51, and one downstream in channel 30. The black substrate method may provide a local (additional) depth to channel 30 of about 0.5-10 μm, preferably 0.5-5 μm, such as 1-2 μm. The “grass” features are preferably 0.5 to 10 μm high.

Hence, according to another aspect of the invention, there is provided a mould or a mask for use in a process according to the invention. Such a mould or mask can comprise any known available mould or material. The mould or mask however, comprise structures such that in a moulding process like hot embossing or LIGA, or in an etching process like lithographic etching, the mask provides the desired channel structure into the body material.

According to a further aspect of the invention, there is provided a method for sampling liquid(s) from the GI tract comprising swallowing sampling device 1.

According to yet another aspect of the invention, there is provided a method for analyzing liquid(s) from the GI tract of a human or an animal comprising swallowing sampling device 1 according to the invention by the human or animal, recovering sampling device 1 from the GI tract of the human or animal (from the stool, or by GI surgery), optionally extracting the liquid from sampling device 1, and analyzing at least part of the liquid sampled by sampling device 1 with analyzing techniques known per se.

Examination of internal body fluids or gases in the digestive system or the gastrointestinal tract in the human or animal body may provide essential medical information for diagnosis and treatment. Examination of samples of the gastric fluid of a patient may provide important information of pH, acid contents, abdominal enzyme activity as well as information for diagnosing gastric ulcer and gastritis, cancer and tumour diseases, etc. An examination with the device according to the invention may give the physician who is treating a patient important information and may play a helpful role in diagnosis. Also, present device 1 may be used as a research tool by gathering information on the presence of species and processes taking place in a certain period of time in a human or animal. It may also provide information on the digestion of known or novel food or feed components, drugs or nutraceuticals and the like. Hence in an embodiment, device 1 may be used for pharmacokinetical, pharmacomimetical, pharmacodynamical, or nutridynamical studies in the human or animal body (see for instance also W. M. de Vos et al., Current Opinion in Biotechnology, 2006 (17), 217-225).

In an embodiment, a vacuum is applied to channel 30, e.g. such that a pressure is obtained equal to or smaller than about 0.8 bar, more preferably equal to or smaller than about 0.01 bar, even more preferably between about 0.01 and 1.10⁻⁵ bar. Then opening 21 may be closed with e.g. a plug or a coating, indicated in FIGS. 5 and 6 with reference number 222. For example, the device is connected to a vacuum pump via opening 21 as long as is necessary to obtain a sufficiently low vacuum (preferably <0.01 bar) inside the chip (for example about 1-180 minutes, depending upon the type of pump, the construction of the channel structure (including possible restrictions), etc.). Subsequently the chip is sealed at opening 21 with coating 222. Then device 1 can be swallowed. In an embodiment, such plug or coating 222 may be made of a material which is chosen depending on the application of device 1, i.e. the specific GI liquid(s) to be sampled. The plug or coating 222 may dissolve after a certain period of time by action of bodily liquid when ingested or after a certain time within the GI tract. Hence, the material of the plug or coating 222 in an embodiment is adapted to the specific GI liquid(s) in the external environment of device 1 where the samples are to be collected. In an embodiment, the material of the plug or coating 222 may for example be selected from one or more of the group consisting of gelatin, sugar, salt, glue, organic edible materials and any other suitable material such as e.g. HPC and/or HPMC (hydrophilic polymer hydroxypropyl (methyl)cellulose) or equivalent materials known to the person skilled in the art. Alternatively, the plug or coating 222 can be made of two or more layers of different materials, which dissolve gradually upon contact with different GI liquids in the digestive system. Nevertheless, opening 21 may also be equipped with other closing means known in the art such as e.g. a radio frequency controlled shutter, a magnetically controlled valve, or the like. Similarly, between optional reservoir 70 and channel 30, for instance in connection channel 74, a radio frequency controlled shutter, a magnetically controlled valve, or the like may be introduced. Such a valve may prevent leaking of quenching liquid or another liquid contained in reservoir 70 to channel 30 when evacuating channel 30 (for example to a pressure below 0.8 bar). Various methods known to the person skilled in the art can be applied in order to obtain an underpressure within channel 30 and optional compartments 50. One method can be providing device 1 into a closed container and applying the desired pressure to the container. Within the container, filter material 22 and plug or coating 222 may be provided to opening 21 of evacuated device 1.

For use of device 1 as sampling device in the GI tract, in an embodiment an assembly 100 comprising at least device 1 and a carrier or a container, such as e.g. a tablet, is used. An example of such a carrier is shown in FIGS. 7a-c, wherein assembly 100 of a carrier 80 and device 1 is schematically shown. In this figure, device 1 comprises a plain channel and a top opening 221 as opening 21. Assembly 100 has a length Lca, a width wca and a height hca (which includes h of device 1, i.e. cover 40 is arranged to body 10, see also FIG. 7b). In an embodiment, the assembly may also be a capsule like assembly. The dimensions of assembly 100 are preferably selected such that device 1 is swallowable. Hence, length Lca is preferably about 3 cm or smaller, e.g. about 0.5-3 cm, and width wca and height hca are preferably about 1.5 cm or smaller, e.g. about 0.2-1.5 cm. In a preferred embodiment, the total volume of assembly 100 is about 0.01-8 cm³. In FIG. 7a, no cover 40 is shown (for the sake of clarity). FIG. 7b shows a front view of assembly 100. In this figure, cover 40 is indicated. In a specific embodiment, cover 40 may be coated with a second carrier material 81, preferably a soluble material, e.g. HPC or HPMC (see also above) arranged such that the assembly is provided with a capsule like structure, as schematically depicted in FIG. 7c. Herein reference number 80 refers to a carrier (not or substantially not soluble in the GI liquids), and reference number 81 refers to a second carrier comprising a soluble material, which dissolves in the GI tract, such that sampling via opening 21 can start. After intake, the soluble top material 81 can dissolve, and an assembly as depicted in FIG. 7a/7b appears in the GI tract (of course including cover 40). Intake of assembly 100 may be more easy in this way. Any type of carrier 100 can be used for providing assembly 100 wherein the carrier 80 (and optional second carrier 81) and device 1 can be arranged such as to provide tablets, capsules, pills with dimensions known in the art, The assembly 100 may further comprise, for example included in or on the carrier or included in or on coating or seal 222 additives such as pharmaceutically acceptable additives e.g., sweeteners, colouring agents, flavouring agents, etc. When device 1 comprises a side opening 21, both carriers 80 and second carrier 81 may be insoluble, e.g. an insoluble capsule with an opening 21.

Further, optional reservoir 70 may be filled with the stabilizing or quenching liquid via opening 71, and opening 71 may be sealed with a permanent seal (not dissolving in the GI liquids). After swallowing device 1 by the person or animal whose or which GI tract is to be investigated, device 1 can start sampling at a predetermined position of the GI tract (predetermined by selecting the seal or coating 222 in or on opening 21, for example depending upon the solubility in GI liquids). After recovering device 1 from the person's or animals' stool, the sampled liquids may be analyzed. In this way, sampling device 1 is in a natural way removed from the GI tract of the human or animal.

Application of vacuum, as described above, may be applied to the channel structure such that vacuum is the main driving force for sampling. A specific method is explained referring to FIGS. 9a and 9c, although this method may also be applied to other embodiments described herein.

In an embodiment, channel 30 further comprises a branch channel, indicated as vacuum facilitation channel 30a. This vacuum facilitation channel 30a preferably comprises at least two assisting openings, which are indicated as openings 21a and 21b, respectively. These openings are only used in the method for creating vacuum in the channel structure. Opening 21 is closed with the herein described closing means 222, which may be for instance be a coating on or in opening 21. Opening 21b is closed with a closing means, preferably a foil or a film, such as a paraffin film. Opening 21a is attached to a vacuum pump, and a vacuum is applied, such as the herein described vacuum. Thereby, the whole channel structure, inclusive optional side compartments 50 and optional side compartment channels 51 is evacuated. Further, a preferably UV hardening resin or glue is applied on the closing means on opening 21b. After having obtained the desired vacuum, the film or foil arranged on opening 21b is pressed into the opening and the glue or resin is hardened by UV light. Some of the glue or resin may flow into channel 30a. In this way, opening 21b is closed and vacuum facilitation channel 30a is closed from the environment. An example of a suitable UV glue is for instance Loctite 385. Hence, during sampling vacuum facilitation channel 30a is closed to maintain the vacuum.

Analysis can be performed in different ways. For example, analysis methods may be applied on the liquid in device 1 without removing the liquid, e.g. by applying optical measuring techniques and using a cover 40 that is at least partially transparent for the optical signal to be detected or the optical radiation used for irradiating at least part of the liquid in channel 30 (plain embodiment) and one or more optional side compartments 50 (compartment embodiment), or at least transparent for both. For example, sample cover(s) 42 for covering side compartments 50 in the compartment embodiment may be of a transparent material (see also above).

Analysis can also be performed on liquid removed from device 1. For example, referring to the plain channel as schematically depicted e.g. in FIG. 1, with a needle in opening 21 at first end 25 liquid may be sucked from channel 30 and may e.g. be divided over one or more analysis vessels for further analysis. However, there may also be provided a second opening at second end 26, either as permeable part of cover 40 over second end 26 of channel 30, or as opening with a closing means, known to the person skilled in the art, that may be removed after sampling the GI liquids in the tract. In another embodiment, two (or more) needles (or similar means) are used, in order to prevent creating a vacuum while extracting (for instance sampled liquid from a side compartment 50). A second needle may also be used to introduce liquid while extracting sampled liquid from a compartment 50, e.g. for “flushing” a compartment 50.

Further, referring to the compartment embodiment as e.g. schematically depicted in FIG. 2 etc., liquid may be removed relatively easily from a compartment 50 by using a needle, e.g. since cover 40 is removed, or since cover 40 is permeable for the needle (e.g. sample compartment cover 42 may be permeable to this end), or since cover 40 comprises parts 45 with reduced height h3, providing permeable parts of cover above compartment(s) 50.

In yet a further embodiment, before analysis, cover 40 may be removed.

Hence, according to an aspect of the invention there is also provided the use of device 1 for the preparation of assembly 100 for sampling of liquid(s) from the gastro-intestinal tract of a human or animal. In yet another aspect, there is provided a method of sampling of liquid(s) from the gastro-intestinal tract comprising administering assembly 100 comprising device 1, a carrier 80 and optional second carrier 81 to a target human or animal. This method may further comprise recovering the assembly from the stool and analysing the one or more samples sampled by device 1. However, device 1 (contained in assembly 100) of the invention may also be arranged stationary in the GI tract.

The time until the channel structure of device 1 is filled, i.e. the sampling time, can be tuned by varying height h1, width w1 and length of channel 30, i.e. the volume of the channel structure, including the volume of optional side compartments 50 and side channels 51 (see below), but also by optional barrier 33 and optional hydrophobic barriers 33a (see above). Depending upon the desired application, the person skilled in the art will choose the appropriate parameters to obtain the desired sampling volume and/or sampling time.

According to yet a further aspect of the invention there is provided a sampling device for sampling liquids, a production process therefore, and mould or mask for use in the production process, in general. The sampling device is suitable for sampling of liquid(s) from one or more selected of body liquids, water and other liquids. Hence, instead of GI liquids, also other liquids may be sampled with the device of the invention. The device comprises a body, the body comprising a channel and an opening for entrance of the liquid(s) at one end of the channel, and a cover bonded to at least part of the body and arranged such that the channel in the body is at least partially covered by the cover. The channel may have a length of 2 mm-25 m and a perimeter of 2.4-8600 g/m, and the channel may further optionally comprise one or more side compartments. The channel with the optional one or more side compartments may have a volume of 5 nl-4500 μl. However, depending upon the desired application, also other dimension will be used, as will be clear to the person skilled in the art.

Hence, the device according to the invention is not only limited to use in the GI tract for sampling GI liquids, but may in general be used and be designed such to sample liquids in other organs of the human or animal body such as the lymph system (for sampling lymphatic fluids), arteries, heart, blood vessels or other human or animal systems for transporting and for sampling blood, etc. The device or an assembly comprising the device may be mobile (for instance when swallowed), but may also be arranged stationary, such as implanted sub-dermal/sub-cutaneous, etc. The device according to the invention may for instance be used in pharmacokinetics etc. (see also above) in humans or animals (such as dogs, like beagle dogs, rodents, like mice or rats, monkeys, etc.). In this way, the influence of drugs or nutrition can be tested, for instance by sampling in the GI channel, but also by sampling liquids in other organs (see above), such as by sampling blood. Hence, in a specific embodiment there is provided a method of sampling of liquid(s) from a target human or animal, comprising administering device 1 (for instance as assembly 100 comprising device 1 and further comprising a carrier 80 and optional second carrier 81) to a target human or animal. This method may further comprise analysing the one or more samples sampled by device 1. In yet a further embodiment, the invention comprises a method for performing a pharmacokinetical, pharmacomimetical, pharmacodynamical, or nutridynamical study in the human or animal body, comprising administering device 1 (for instance as assembly 100 comprising device 1 and further comprising a carrier 80 and optional second carrier 81) to a target human or animal and analysing the liquid(s) sampled by the device 1. The person skilled in the art knows how to perform such study, for instance by further administering nutrition or drugs to the target human or animal. The impact of the nutrition and/or drug on the human or animal body may then be evaluated by analysing the sampled liquid(s), such as blood, GI liquids, etc.

The device according to the invention may also be used to sample liquids (water or other liquids) in man made systems such as industrial systems like (bio)reactors, pipings, supply vessels, waste water treatment plants, sewage pipings, water supply systems, aquaria and piping everywhere, but also for sampling liquids in natural ecosystems such as marine and fresh water systems, ground water systems, or other systems like oil or petroleum transport pipes, etc.

The person skilled in the art will adapt the dimensions of the channel, optional compartments, opening(s) etc. from the channel structure, as well as the dimensions of the complete device 1 or of an assembly comprising device 1, for use in these (non-GI) applications. Hence, the dimensions of a device for use in such applications may differ from the dimensions which are suitable for application of the device as sampling device for sampling GI liquids. For other applications than in the GI tract, other assemblies, if necessary, can be used. Further, device 1 or assembly 100 may be arranged stationary, for instance in a waste stream or a stream of sewage, oil or petroleum pipe, etc. Pollution or composition as a function of time may be monitored in this way. However, the device of the invention may also be arranged stationary, sampling liquid(s) that pass.

### EXAMPLES

### Example 1

### Si Etching

Here an example is given of Si etching. Specific conditions and parameters of a general Si etching process are provided as examples.

In a clean room environment, a silicon wafer with a 100 mm diameter and a thickness of 525 μm is provided.

In a next step, cleaning is performed in order to remove possible organic contamination using fuming nitric acid. Then a second cleaning is performed in order to remove e.g. native oxides and metals, by applying a HF dip. In this way, a clean hydrophobic silicon surface of the Si wafer is obtained.

Then, step spinning of HMDS (hydrophobic) is performed and a photo resist (such as Olin907/17) is subsequently spun onto the surface such that a thickness of 1.6 μm or 3.5 μm is obtained.

A further step comprises a prebake, for e.g. 1 min. at 95° C.

Subsequently, the photo resist is exposed, e.g. for 5 seconds with UV radiation of 325 nm, using a predesigned mask to provide the channel structure of the invention.

Then, the Si wafer with exposed resist is developed, e.g. dipping for 60 seconds in a beaker containing photoresist developer.

After an optional optical inspection (checking e.g. accuracy, developing), a post bake may be performed, e.g. 30 min. at 95° C.

Then, silicon etching may be performed, e.g. with an Adixen etching set up using a Bosch process. For example, only 20 min. are necessary for about 100 μm. The Bosch process with the Adixen is cooled at 10° C., and may etch about 5 μm/min, using a high vacuum, gas flow, plasma ignition, auto pressure control for constant plasma pressure, etc. The Bosch process is a double operating process with polymer deposition at sidewalls and etching by plasma enhanced reactive ion etching.

In the next step, an oxygen plasma is provided in order to remove the photo resist.

Further, a “Piranha” (90° C.; H₂SO₄/H₂O₂) cleaning step is performed in order to remove the last remains of the photo resist and to remove any possible precipitate.

Then, cover 40 may be bonded to etched Si wafer 10, e.g. by anodic bonding at conditions such as 400° C. and 1000V.

Finally, using e.g. a water cooled (circle) saw, device 1 (or a number of devices 1) is derived from the wafer.

### Example 2

### Plain Channel Device

According to the method described above and using a 525 μm Si wafer, device 1 is provided with plain channel 30 (see e.g. FIGS. 1 and 8a) having a length of 2 meter, a width w1 of 30 μm, a channel height or depth h1 of 300 μm. The total volume of channel 30 is 18 μl. The above channel structure is provided in the Si wafer and after sawing the wafer, device 1 with this channel structure is provided. This device 1 has a length L of 17 mm and a width w of 8 mm. The height h is 1025 μm (h4=525 μm; h2=500 μm).

According to the method describe above and using a 525 μm Si wafer, device 1 is provided with channel 30 with compartments 50 (see e.g. FIG. 2), channel 30 having a length of 0.08 m meter, a width w1 of 30 μm, a channel height or depth h1 of 2 μm. The total volume of channel 30 is 4.8 nl. 96 compartments 50 are provided, each having a diameter (width wdc) of 500 μm, a depth/height dc of 300 μm, and a volume of each compartment of 0.06 μl. The total volume of all 96 compartments 50 is 6 μl. After each intersection between channel and compartment, i.e. after each side channel 51, channel 30 is made partially hydrophobic by treatment with a hydrophobicity inducing or enhancing means. Such means is applied through a patterned masking film, created with standard lithograph technology. In this example, FDTS (1H,1H,2H,2H-perfluorodecyltrichlorosilane) and a patterned aluminium masking layer were applied. The FDTS reaction is performed in a low-pressure reaction vessel for 1 hour at RT (room temperature). The obtained perfluorodecyl coating is annealed for 5 minutes at 150° C. The hydrophobic channel section can differ in length from 1 μm up to the distance to the next channel/compartment intersection. The above channel structure is provided in the Si wafer and after sawing the wafer, device 1 with this channel structure is provided. This device 1 has a length L of 17 mm and a width w of 8 mm and the height h is 1025 μm.

In a specific embodiment, compartments 50 have a width wdc of about 500 μm, channel 30 has a length of 70 mm and the depth h1 of channel 30 is 3 μm.

### Example 4

### Analysis of Microbial Metabolite Production in the GI Tract

According to the method described above samples can be taken (as a function of time) from the GI tract of a human or animal. In a preferred embodiment sampling may start in the large intestine, and samples collected will contain microbial metabolites produced by the intestinal microbiota. For instance, the collected samples may contain short chain fatty acids (SCFA; acetate, propionate, butyrate, valerate). After collection of the individual samples from the side-compartment, these samples may be measured with Fourier Transform Mass Spectrometry (FT-MS) or other MS methods to identify the presence and concentration of SCFA. Especially the presence and concentration of butyrate is interesting, as this is the preferred fuel of colonocytes, the epithelial cells lining the large intestine.

### Example 5

### Device for GI Sampling

Referring to FIG. 7c, an assembly 100 like a capsule can be provided, with dimensions that assembly 100 is swallowable. Hence, length Lca (see FIG. 7a) may be 3 cm and height hca (i.e. in this case the diameter) is 1.5 cm or smaller (for instance 1 cm).

### Example 6

### Sampling Test with Colour Liquids

A sampling device 1, similar to the in FIG. 9c schematically depicted embodiment, based on silicon and including a glass cover 40 was made according to the invention. The length L was 18 mm, the width W was 9 mm. The channel width w1 was 100 μm; the channel depth h1 and the side compartment channel height were both 4 μm (preferably, the channel depth h1 and the side compartment channel height are substantially equal). Just behind each bifurcation into channel 30 and side compartment channel 51, a hydrophobic barrier 33a was arranged, as indicated with reference number 33a in FIG. 9d. The length of these hydrophobic barriers 33a in channel 30 are about 100 μm, respectively. Preferred lengths are 10-500 μm, more preferably 20-150 μm. The depth of the black silicon, i.e. the additional depth between the grass blades of the hydrophobic barrier 33a relative to channel 30 is about 2 μm. The side compartment height dc was 100 μm, and the diameter wdc was 800 μm.

The device 1 was evacuated to a pressure of about 1 mbar. Opening 21 was brought into contact with water containing an ink. After a while, the colour of the water was changed by introducing water with a different colour in the vessel from which the device samples the liquid through opening 21. When all side compartments 50 were filled with coloured water, it was apparent that the compartments 50 close to opening 21, i.e. close to first end 25 had the colour of the starting liquid and the compartments close to second end 26 had the colour of the liquid at the end of the sampling period. The colour change of the liquids in the compartments 50 reflect the colour change in time of the colour of the water that was sampled by sampling device 1.

### Example 7

### Applying a Black Silicon Hydrophobic Barrier

In a compartment sampling device (according to example 3) a pattern in the existing channel was provided using a masking film, created with standard lithography technology. The actual etching was performed in an Adixen AMS 100SE reactive ion etcher, using fast alternating (1/0.2 sec) plasmas of SF₆ (500 sccm) and C₄F₈ (200 sccm). “Black silicon” was produced after 5 min of etching.

Hydrophobicity was measured in an optical contact angle measuring device and was determined at probably larger than 175° on a large area of “black silicon” on a silicon wafer.

The embodiments described above and as schematically depicted in the drawings are not to scale. Further, only those features relevant for the invention are depicted and described. The scope of protection of the invention is not limited to the embodiments given. The invention resides in each novel characteristic and each combination of characteristics. Reference numerals in the claims do not limit the scope of protection thereof. The use of the verb “comprise” and its declinations does not exclude the presence of elements other than those specified in the claims. The use of the indefinite article “a” or “an” preceding an element does not exclude the presence of a plurality of such elements. The term “about” before a numerical value indicates that the “exact” numerical value may be used, but that also values slightly deviating (e.g. less than 10%, more preferably less than 5%, and even more preferably less than 1% difference relative to the value) may be applied.

Reference number list

| | |
|---|---|
| L | length device 1 |
| Lca | length of assembly 100 |
| w | width device 1 |
| h | height device 1 |
| w1 | width of channel 30 |
| h1 | height of channel 30 |
| h2 | height of cover 40 |
| h3 | thickness/height of cover 40 |
| | with reduced thickness |
| h4 | height of body 10 |
| hca | height of assembly 100 |
| dc | depth of compartment 50 |
| wdc | diameter of compartment 50 |
| wca | width of assembly 100 |
| t₀ | starting time of sampling GI |
| | liquid |
| t_{end} | last moment of sampling |
| 1 | sampling device |
| 10 | body of device 1 |
| 21 | opening to channel 30 |
| 21a,b | forst (a) and second (b) |
| | opening in vacuum facilitation |
| | channel |
| 22 | filter material |
| 24 | opening compartment |
| 25 | first end of channel 30 |
| 26 | second end of channel 30 |
| 30 | channel |
| 30a | vacuum facilitation channel |
| 30b | bottom surface of channel 30 |
| 33 | barrier in channel 30 |
| 33a | hydrophobic barrier |
| 36 | flow direction |
| 40 | cover of device 1 |
| 41 | glue |
| 42 | sample cover |
| 43 | fixation means |
| 45 | part of cover with reduced |
| | height |
| 50 | side compartment |
| 50b | bottom surface of side |
| | compartment 50 |
| 50(1) | example of compartment 50 |
| 50(2) | example of compartment 50 |
| 50(3) | example of compartment 50 |
| 51 | side compartment channel |
| 52 | upstream (relative to a side |
| | compartment 50(1)) |
| 53 | downstream (relative to a side |
| | compartment 50(1)) |
| 60 | supporting means for filter |
| | material 22 |
| 70 | resevoir for stabilizing liquid |
| 71 | opening to resevoir 70 |
| 72 | resevoir filling channel |
| 73 | barrier in connecting channel |
| | 74 |
| 74 | connecting channel |
| 80 | carrier |
| 81 | second carrier |
| 95 | a position in beginning of |
| | channel |
| 96 | liquid front in channel |
| 100 | assembly of device 1 and |
| | carrier 80 |
| 221 | top opening in device 1 |
| 222 | plug or coating on or in |
| | opening 21 |

1. A sampling device for in vivo sampling of a liquid, comprising a body, the body comprising a main channel with an opening for entrance of the liquid at a first end of the main channel, and a cover bonded to at least part of the body and arranged such that the main channel is at least partially covered by the cover, wherein the main channel has a length of 2 mm-25 m and a perimeter of 2.4 μm-8600 μm.

2. The device according to claim 1, wherein the liquid is gastro-intestinal tract liquid.

3. The device according to claim 1, wherein the main channel further comprises one or more side compartments.

4. The device according to claim 3, wherein the main channel with one or more side compartments has a volume of 5 nl-4500 μl.

5. The device according to claim 1, wherein at least part of the body comprises a material selected from the group consisting of silicon, glass, fused silica, quartz, ceramic and plastic.

6. The device according to claim 3, wherein the one or more side compartments has a volume ranging from 2 nl-1.5 μl.

7. The device according to claim 3 comprising 2 or more side compartments.

8. The device according to claim 7 comprising 10 or more side compartments.

9. The device according to claim 3 wherein the main channel has a length of 0.01-0.2 m, a width of 10-100 μm and a depth of 0.5-5 μm, and one or more side compartments having a volume of 10-1000 nl.

10. The device according to claim 1, wherein the sampling device is arranged to have a liquid flow from the first end of the main channel to the other end of the main channel, wherein the main channel comprises a plurality of side compartments connected to the main channel via side compartment channels, wherein the main channel comprises an upstream part and a downstream part relative to the side compartment channels, and wherein the side compartment channels are arranged to be substantially in line with the flow direction in the upstream parts of the main channel.

11. The device according to claim 3,
a. wherein the device comprises a first compartment, and wherein the main channel has an upstream part and a downstream part relative to the side compartment channel to the first side compartment;
b. wherein the device further comprises a second compartment, and wherein the main channel has an upstream part and a downstream part relative to the side compartment channel to second side compartment;
c. wherein relative to a liquid flow in the main channel from the first end in the direction of the second end first side compartment is arranged upstream relative to the second side compartment;
d. and wherein a flow resistance for the liquid to flow from the upstream part into the side compartment channel to the first side compartment is substantially smaller than a flow resistance for the liquid to flow from the upstream part into the upstream part (of the main channel.

12. The device according to claim 1, wherein the main channel further comprises a plurality of side compartments connected to the main channel via side compartment channels, and wherein in the main channel comprises hydrophobic barriers that direct the flow of the liquid in the direction of the side compartments.

13. The device according to claim 12, wherein the hydrophobic barriers comprise black silicon.

14. The device according to claim 11, wherein the hydrophobic barriers have a contact angle of at least 130°.

15. The device according to claim 12, wherein the hydrophobic barriers have a contact angle for water of at least 130°.

16. The device according to claim 1, wherein each side compartment has a volume of about 2 nl-1.5 μl.

17. A sampling device for in vivo sampling of a liquid, comprising:
a body, the body comprising a channel with an opening for entrance of the liquid at a first end of the channel, wherein the channel has a length of 2 mm-25 m and a perimeter of 2.4-8600 μm;
a cover bonded to at least part of the body and arranged such that channel is at least partially covered by the cover; and
one or more side compartments having a volume of 5 nl-4500 μl,
wherein the device is arranged to sample the liquid(s) into the channel and the one or more side compartments substantially by means of a vacuum force.

18. The device according to claim 1, wherein the liquid is gastro-intestinal tract liquid.

19. The device according to claim 17, wherein the channel further comprises hydrophobic barriers that direct flow of the liquid in the direction of the side compartments.

20. The device according to claim 1, wherein the device is arranged to sample the liquid by means of vacuum force.

21. A process for the production of a sampling device for sampling a liquid, comprising:
a. providing a channel having an opening for entrance of the liquid at one end of the channel in a body, the channel having a length of 2 mm-25 m and a perimeter of 2.4-8600 μm, and
b. binding a cover to at least part of body such that channel of the body is at least partially covered by the cover.

22. The process according to claim 21, wherein the liquid is gastro-intestinal tract liquid.

23. The process according to claim 21, further comprising:
c. providing one or more side compartments and
d. operably coupling the one or more side compartments to the channel.

24. The process according to claim 23, wherein the one or more side compartments have a volume of 5 nl-4500 μl

25. The process according to claim 24, wherein the coupling step is performed by a process selected from the group consisting of an etching process, a hot embossing process and a LIGA process.

26. A mould or a mask for use in a process according to claim 25.

27. A method of sampling a liquid from a human or animal body in vivo comprising administering a device comprising a body, the body comprising a main channel with an opening for entrance of the liquid at a first end of the main channel, and a cover bonded to at least part of the body and arranged such that the main channel is at least partially covered by the cover, wherein the main channel has a length of 2 mm-25 m and a perimeter of 2.4 μm-8600 μm.

28. The method according to claim 27, wherein the liquid is a GI liquid, lymph liquid, or blood.

29. A method of sampling a liquid from a human or animal body in vivo comprising administering a device comprising a body a body, the body comprising a channel with an opening for entrance of the liquid at a first end of the channel, wherein the channel has a length of 2 mm-25 m and a perimeter of 2.4-8600 μm;
a cover bonded to at least part of the body and arranged such that channel is at least partially covered by the cover; and
one or more side compartments having a volume of 5 nl-4500 μl,
wherein the device is arranged to sample the liquid(s) into the channel and the one or more side compartments substantially by means of a vacuum force.

30. The method according to claim 29, wherein the liquid is a GI liquid, lymph liquid, or blood.

31. A method of sampling liquids in systems selected from the group consisting of (bio)reactors, pipings, supply vessels, waste water treatment plants, sewage pipings, water supply systems, aquaria, marine systems, and fresh water systems, the method comprising administering a device comprising a body, the body comprising a main channel with an opening for entrance of the liquid at a first end of the main channel, and a cover bonded to at least part of the body and arranged such that the main channel is at least partially covered by the cover, wherein the main channel has a length of 2 mm-25 m and a perimeter of 2.4 μm-8600 μm.

32. The method according to claim 27, wherein the administration comprises subcutaneous implantation.

33. The method according to claim 29, wherein the administration comprises subcutaneous implantation.

34. A method for performing a pharmacokinetical, pharmacomimetical, pharmacodynamical, or nutridynamical study in the human or animal body, comprising administering a device comprising a body, the body comprising a main channel with an opening for entrance of the liquid at a first end of the main channel, and a cover bonded to at least part of the body and arranged such that the main channel is at least partially covered by the cover, wherein the main channel has a length of 2 mm-25 m and a perimeter of 2.4 μm-8600 μm.

35. A method for performing a pharmacokinetical, pharmacomimetical, pharmacodynamical, or nutridynamical study in the human or animal body, comprising administering the device according to claim 11 to a target human or animal and analysing the liquid(s) sampled by the device.

36. A method of sampling of liquid(s) from the gastro-intestinal tract comprising administering an assembly comprising a device comprising a body, the body comprising a main channel with an opening for entrance of the liquid at a first end of the main channel, and a cover bonded to at least part of the body and arranged such that the main channel is at least partially covered by the cover, wherein the main channel has a length of 2 mm-25 m and a perimeter of 2.4 μm-8600 μm; and a carrier to a target human or animal.
